(19) United States
(12) Patent Application Publication
(10) Pub. No.: US 2025/0139775 A1
(43) Pub. Date: May 1, 2025

(54) IMAGE SEGMENTATION SYSTEM VIA GRAPH OR MULTISCALE CASCADED ATTENTION DECODING

(71) Applicant: BOARD OF REGENTS, THE UNIVERSITY OF TEXAS SYSTEM, Austin, TX (US)

(72) Inventors: Radu Marculescu, Austin, TX (US); Mostafijur Rahman, Austin, TX (US)

(21) Appl. No.: 18/929,153

(22) Filed: Oct. 28, 2024

Related U.S. Application Data
(60) Provisional application No. 63/593,726, filed on Oct. 27, 2023.
(60) Provisional application No. 63/711,588, filed on Oct. 24, 2024.

(51) Int. Cl. G06T 7/00; G06T 7/10; G06V 10/80; G16H 50/20; H04N 19/176

(52) U.S. Cl. CPC G06T 7/0012; G06T 7/10; G06V 10/806; G16H 50/20; H04N 19/176; G06T 2207/20084; G06T 2207/30096

(57) ABSTRACT

An exemplary image segmentation method and system that employs, in a deep neural network, (i) an attention gate that fuses features with skip connections and (ii) a graph or multi-scale convolutional attention module that enhances the long-range and local context. The segmented region or image data derived in part from the segmented region can be subsequently employed for diagnosis, controls, planning, assessment, or analysis of various diseases. The image data can be medical images (e.g., from medical instruments), sensor images or videos (e.g., from smart phones, cameras, satellites, etc.), as well as 3D objects or volume.

### RELATED APPLICATION

This U.S. application claims priority to, and the benefit of, U.S. Provisional Patent Application No. 63/593,726, filed Oct. 27, 2023, entitled “Image Segmentation via Cascaded Attention Decoding,” and U.S. Provisional Patent Application No. 63/711,588, filed Oct. 24, 2024, entitled “Image Segmentation via Cascaded Attention Decoding,” each of which is incorporated by reference herein in its entirety.

### BACKGROUND

Image segmentation involves partitioning objects, boundaries, or structures within the image into groups of pixels to extract meaningful information and teach computers to perceive and understand visual data in a manner that humans understand, view, and perceive.

Image segmentation has become an important application in the field of computer-aided diagnosis, where resources are limited. The state-of-the-art image segmentation systems and methods require a lot of computational power and resources to perform segmentation, and the segmented images or data can come with low-quality.

There would be, therefore, a benefit to improving the image segmentation systems and methods.

### SUMMARY

An exemplary image segmentation method and system are disclosed (also referred to as graph Cascaded Attention Decoder (G-CASCADE) or Efficient Multi-scale Convolutional Attention Decoding (EMCAD) that employs, in a deep neural network, (i) an attention gate that fuses features with skip connections and (ii) a graph or multi-scale convolutional attention module that enhances the long-range and local context. Experimentation demonstrated that a transformer with G-CASCADE and EMCAD configuration can significantly outperform state-of-the-art CNN—and existing transformer-based approaches. The segmented region or image data derived in part from the segmented region can be subsequently employed for diagnosis, controls, planning, assessment, or analysis of various diseases. The image data can be medical images (e.g., from medical instruments), sensor images or videos (e.g., from smart phones, cameras, satellites, etc.), as well as 3D objects or volume.

The convolutional attention module can enhance the long-range and local context. The decoder can use a multi-stage feature and loss aggregation operation that ensures faster convergence and better performance. The exemplary method and system can improve the accuracy of medical image segmentation, e.g., for pretreatment diagnosis, treatment planning, and post-treatment assessments of various diseases. The G-CASCADE decoder can enhance the feature maps by preserving long-range attention due to the global receptive field of the graph convolution operation while incorporating local attention through the spatial attention mechanism.

The EMCAD decoder employs a multi-scale depth-wise convolution block and can enhance the feature maps via efficient multi-scale convolutions while incorporating complex spatial relationships and local attention through the use of channel, spatial, and grouped (large-kernel) gated attention mechanisms.

The G-CASCADE-based decoder and EMCAD-based decoder can be used with other hierarchical vision encoders for feature refinement and decoding.

In an aspect, a method is disclosed comprising receiving, by a processor, a set of one or more image data; and determining, by a processor, a segmented region within at least one of the image data of the set of one or more image data using a deep neural network (e.g., CNN, CNN-based, transformers) configured with i) an attention gate that fuses features with skip connections and ii) a convolutional attention component, wherein the segmented region or an image data derived from use of the segmented region is subsequently employed for diagnosis, controls, planning, assessment, or analysis.

In another aspect, a method is disclosed comprising: receiving, by a processor, a set of one or more image data (e.g., image or video); and determining, by the processor, a segmented region within at least one of the image data of the set of one or more image data using a deep neural network (e.g., CNN, CNN-based, transformers) configured as a cascading transformer comprising (i) encoder configured with a plurality of encoding blocks arranged in a cascading manner and (ii) decoding blocks each comprising an attention gate that fuses features with skip connections from a corresponding encoder block and at least graph or multi-scale convolutional attention components, wherein the segmented region or an image data derived from use of the segmented region is subsequently employed for diagnosis, controls, planning, assessment, or analysis.

In some embodiments, the decoding blocks, as a part of a graph convolutional decoder, are configured with the graph convolutional attention components (e.g., Graph convolutional attention module (GCAM)) employing at least one or more graph convolution layers.

In some embodiments, the graph convolutional attention component includes the at least one or more graph convolution layers connected to one or more convolution layers.

In some embodiments, each graph convolutional attention component includes a graph convolution block and a spatial attention module.

In some embodiments, to aggregate the multi-scale features, each decoder block is configured to (i) upsample features from a previous decoder block with the features from a skip connection connected to the corresponding encoder block to generate combined upsampled features and (ii) direct the combined upsamples features to the decoding blocks, wherein each output of each stage of the decoding blocks are combined in a convolution layer (e.g., segmentation/prediction head).

In some embodiments, the decoding blocks, as a part of a multi-scale convolutional decoder, are configured with the multi-scale convolutional attention components (e.g., MSCAM) employing at least one or more multi-scale convolution layers.

In some embodiments, the multi-scale convolutional attention component includes the at least one or more multi-scale convolution layers connected to one or more convolution layers.

In some embodiments, each multi-scale convolutional attention component includes a multi-scale convolution block, a channel attention module, and a spatial attention module.

In some embodiments, to aggregate the multi-scale features, each decoder block is configured to (i) upsample features from a previous decoder block with the features from a skip connection connected via a group attention gate (e.g., LGAG) to the corresponding encoder block to generate combined upsampled features and (ii) direct the combined upsamples features to the decoding blocks, wherein each output of each stage of the decoding blocks are combined in a convolution layer (e.g., segmentation/prediction head).

In some embodiments, the set of one or more image data comprises medical images (e.g., ultrasound, CT, MRI, endoscopy, OCT), and wherein the segmented region is subsequently employed for pretreatment diagnosis, treatment planning, and/or post-treatment assessments of a disease (e.g., to generate segmentation maps of lesions or organs).

In some embodiments, the deep neural network forms a hierarchical cascaded attention-based decoder.

In some embodiments, the segmented region or image data derived from the use of the segmented region is employed in a control application (e.g., real-time control application) or for image analysis (e.g., in an image analysis toolkit).

In some embodiments, the set of one or more image data are 2D images, 3D objects (e.g., volumetric objects), or 4D images or objects (3D images or objects+time).

In another aspect, a system is disclosed comprising: a processor; and a memory having instructions stored thereon, wherein execution of the instructions by the processor causes the processor to: receive a set of one or more image data (e.g., image or video); and determine a segmented region within at least one of the image data of the set of one or more image data using a deep neural network (e.g., CNN, CNN-based, transformers) configured as a cascading transformer comprising (i) encoder configured with a plurality of encoding blocks arranged in a cascading manner and (ii) decoding blocks each comprising an attention gate that fuses features with skip connections from a corresponding encoder block and at least graph or multi-scale convolutional attention components, wherein the segmented region or an image data derived from the use of the segmented region is subsequently employed for diagnosis, controls, planning, assessment, or analysis.

In some embodiments, the decoding blocks, as a part of a graph convolutional decoder, are configured with the graph convolutional attention components (e.g., graph convolutional attention module) employing at least one or more graph convolution layers.

In some embodiments, the graph convolutional attention component includes the at least one or more graph convolution layers connected to one or more convolution layers.

In some embodiments, each graph convolutional attention component includes a graph convolution block and a spatial attention module.

In some embodiments, the decoding blocks, as a part of a multi-scale convolutional decoder, are configured with the multi-scale convolutional attention components (e.g., MSCAM) employing at least one or more multi-scale convolution layers.

In some embodiments, the multi-scale convolutional attention component includes the at least one or more multi-scale convolution layers connected to one or more convolution layers.

In some embodiments, each multi-scale convolutional attention component includes a multi-scale convolution block, a channel attention module, and a spatial attention module.

In another aspect, a non-transitory computer-readable medium is disclosed having instructions stored thereon, wherein execution of the instructions by a processor causes the processor to: receive a set of one or more image data (e.g., image or video); and determine a segmented region within at least one of the image data of the set of one or more image data using a deep neural network (e.g., CNN, CNN-based, transformers) configured as a cascading transformer comprising (i) encoder configured with a plurality of encoding blocks arranged in a cascading manner and (ii) decoding blocks each comprising an attention gate that fuses features with skip connections from a corresponding encoder block and at least graph or multi-scale convolutional attention components, wherein the segmented region or image data derived from use of the segmented region is subsequently employed for diagnosis, controls, planning, assessment, or analysis.

### BRIEF DESCRIPTION OF DRAWINGS

FIGS. 1A, 1B, and 1C each shows an example system for image processing configured with an encoder-decoder deep neural network where the decoder is configured as a hierarchical cascaded attention-based decoder in accordance with an illustrative embodiment. FIG. 1A employs convolution attention components. FIG. 1B employs graph convolution attention components. FIG. 1C employs multi-scale convolution attention components.

FIGS. 2A and 2B each shows an example operation for the convolution attention components of FIGS. 1A, 1B, and 1C in accordance with an illustrative embodiment.

FIG. 3A shows an example encoder-decoder deep neural network where the decoder is configured as a hierarchical cascaded attention-based decoder with graph convolution attention components in accordance with an illustrative embodiment.

FIG. 3B shows an example encoder-decoder deep neural network where the decoder is configured as a hierarchical cascaded attention-based decoder with multi-scale convolution attention components in accordance with an illustrative embodiment.

FIGS. 4A and 4B show an example encoder-decoder deep neural network where the decoder is configured as a hierarchical cascaded attention-based decoder with convolution attention components in accordance with an illustrative embodiment. FIG. 4A shows the hierarchical cascaded attention-based decoder with a cascaded backbone. FIG. 4A shows the hierarchical cascaded attention-based decoder with a parallel backbone.

FIG. 5 shows the segmentation outputs of the G-CASCADE-based systems (e.g., PVT-GCASCADE, MERIT-GCASCADE) and 3 other state-of-the-art methods (e.g., PVT-CASCADE, TransCASCADE, Cascaded MERIT) on 2 sample images.

FIGS. 6A-6C show the quantitative and qualitative results of the EMCAD-based systems (e.g., PVT-EMCAD-B0, PVT-EMCAD-B2) and state-of-the-art methods (e.g., UNet, UNet++, AttnUNet, DeepLabv3+, PraNet, CaraNet, UACANet-L, SSFormer-L, PolypPVT, TransUNet, SwinUNet, TransFuse, UNeXt, PVT-CASCADE, etc.) on datasets (e.g., Polyp, Synapse, ClinicDB). FIG. 6A shows the quantitative results of the EMCAD-based systems and state-of-the-art methods. FIGS. 6B-6C show the qualitative results of the EMCAD-based systems and state-of-the-art methods on datasets.

### DETAILED DESCRIPTION

Some references, which may include various patents, patent applications, and publications, are cited in a reference list and discussed in the disclosure provided herein. The citation and/or discussion of such references is provided merely to clarify the description of the disclosed technology and is not an admission that any such reference is “prior art” to any aspects of the disclosed technology described herein. In terms of notation, “[n]” corresponds to the nth reference in the list. For example, [1] refers to the first reference in the list. All references cited and discussed in this specification are incorporated herein by reference in their entirety and to the same extent as if each reference was individually incorporated by reference.

### Example System

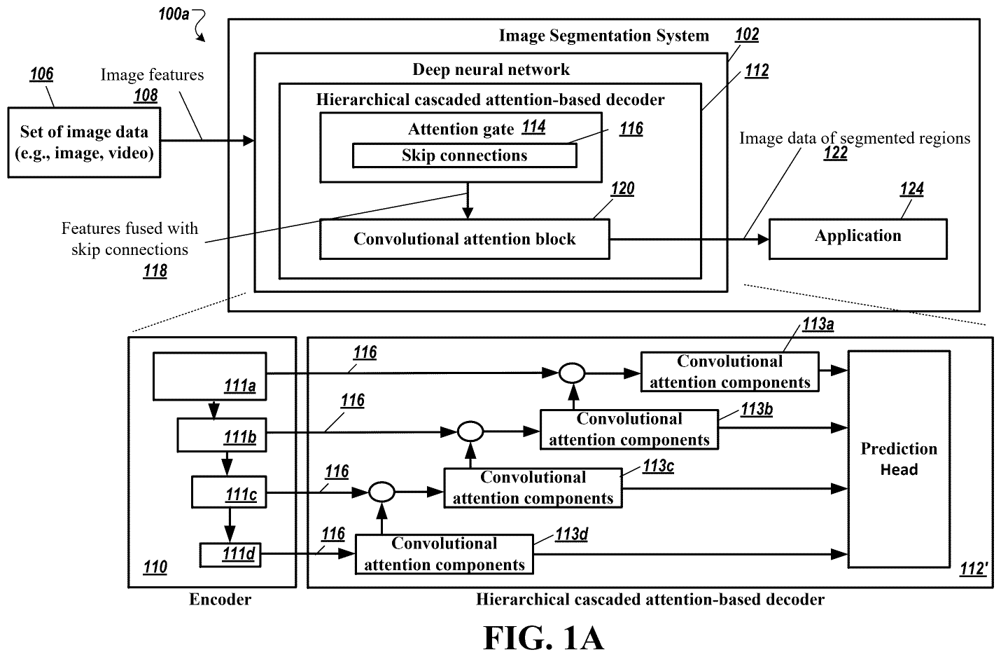

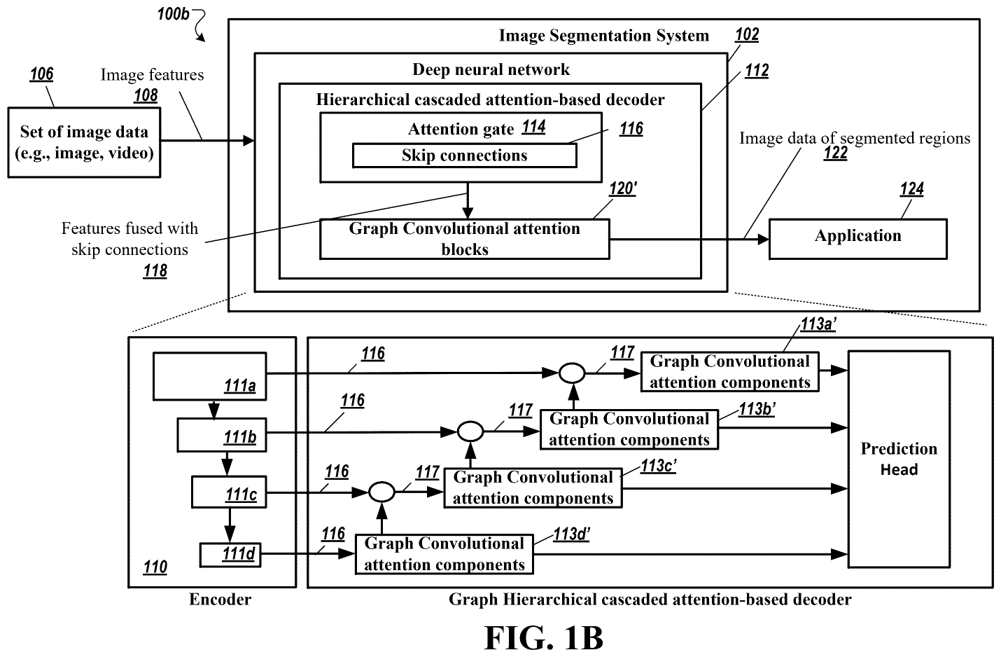

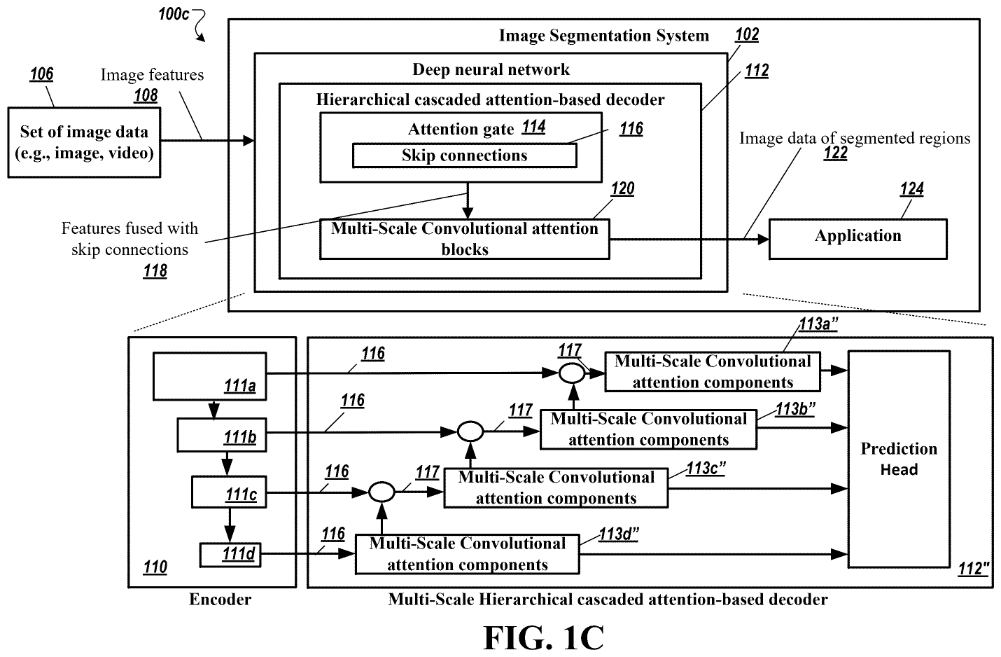

FIGS. 1A, 1B, and 1C each shows an example system 100 (shown as 100a, 100b, 100c) for image processing configured with an encoder-decoder deep neural network where the decoder is configured as a hierarchical cascaded attention-based decoder in accordance with an illustrative embodiment. FIG. 1A employs convolution attention components. FIG. 1B employs graph convolution attention components. FIG. 1C employs multi-scale convolution attention components.

In the example shown in FIG. 1A, the image segmentation system 100a includes a deep neural network 102 for an image processing application 104. The deep neural network 102 is configured with an attention gate module 114 and convolutional attention block 120. The attention gate module 114 and convolutional attention blocks 120 may collectively include a graph convolutional attention module (e.g., Graph convolutional attention module (GCAM), graph convolution block (GCB), spatial attention block (SPA, SAB), efficient up-convolution block (UCB, EUCB), segmentation head (SegHead, SH), large-kernel grouped attention gate (LGAG), multi-scale convolutional attention module (MSCAM), multi-scale convolution block (MSCB), channel attention block (CAB). The execution of the instructions stored on the memory coupled with the processor 104 causes the processor to perform corresponding actions via the neural network 102 as subsequently shown.

The exemplary system can efficiently enhance the feature maps derived from scanning images by preserving long-range attention due to the global receptive field of a graph convolution or multi-scale convolution operation while incorporating local attention through a spatial attention mechanism. Additionally, the exemplary system can also use multi-scale depth-wise convolution blocks to to maximize the performance and computational efficiency while performing image segmentation.

The exemplary system can employ a multi-scale depth-wise convolution block and can enhance the feature maps via efficient multi-scale convolutions while incorporating complex spatial relationships and local attention through the use of channel, spatial, and grouped (large-kernel) gated attention mechanisms.

The deep neural network 102, employed by the exemplary system 100, can receive image features 108 from a set of image data 106 (e.g., image, video). The set of image data 106 can comprise medical images (e.g., ultrasound, CT, MRI, endoscopy, OCT), wherein the medical images can be two-dimensional (2D), three-dimensional (3D), or four-dimensional (4D). The deep neural network 102 includes an encoder 110 and a decoder 112 (shown as “Hierarchical cascaded attention-based decoder” 112′). The encoder 110 is configured with a plurality of encoding blocks 111 (shown as 111a, 111b, 111c, 111d) arranged in a cascading configuration.

The attention gate 114, operating on the neural network 102, is configured to receive outputs from corresponding skip connections 116 of the encoding blocks 111a, 111d. While shown with 4 cascading blocks, the encoder 110 and decoder 112 may each have 2 cascading blocks, 3 cascading blocks, 4 cascading blocks, 5 cascading blocks, 6 cascading blocks, 7 cascading blocks, 8 cascading blocks, 9 cascading blocks, 10 cascading blocks, 11 cascading blocks, 12 cascading blocks, 13 cascading blocks, 14 cascading blocks, 15 cascading blocks, 16 cascading blocks, 17 cascading blocks, 18 cascading blocks, 19 cascading blocks, 20 cascading blocks, 21 cascading blocks, 22 cascading blocks, 23 cascading blocks, 24 cascading blocks. In some embodiments, the encoder 110 and decoder 112 may each have more than 24 cascading blocks. In some embodiments, the number encoder block 111 may be different from that of the decoding block 114 (shown as convolutional attention components 113a, 113b, 113c, 113d). The

The convolutional attention blocks 120, coupled with the attention gate 114, can receive and refine the features fused with skip connections 118. Then, the convolutional attention components generate image data of segmented regions 122 using the refined features. The coupling of the attention gate 114 and the convolutional attention block 120 on the neural network 102 can form a hierarchical cascaded attention-based decoder 112. The application 124, coupled with the convolutional attention block 120, receive the image data of segmented regions 122 and use the image data 122 for diagnosis, controls, planning, assessment, or analysis.

The set of one or more image data 106 may include medical images (e.g., ultrasound, CT, MRI, endoscopy, OCT). The segmented region may subsequently be employed for pretreatment diagnosis, treatment planning, and/or post-treatment assessments of a disease (e.g., to generate segmentation maps of lesions or organs).

The segmented region or image data derived from the use of the segmented region may be employed in a control application (e.g., real-time control application) or for image analysis (e.g., in an image analysis toolkit).

The set of one or more image data may be 2D images, 3D objects (e.g., volumetric objects), or 4D images or objects (3D images or objects+time). In some embodiments, the images are medical images (e.g., CT, MRI, endoscopy, OCT, and ultrasound, among others described or referenced herein). In some embodiments, the images are scientific images (e.g., from microscopes or satellite images). In some embodiments, the images are sensor images or videos (e.g., smartphones or video cameras).

Graph Hierarchical Cascaded Attention-based Decoder. In the example shown in FIG. 1B, the decoding blocks 113 (shown as 113a′, 113b′, 113c′, and 113d′), as a part of a graph convolutional decoder, are configured with the graph convolutional attention components (e.g., Graph convolutional attention module) employing at least one or more graph convolution layers.

In some embodiments, the graph convolutional attention blocks 120 (shown as graph convolutional attention blocks 120′) includes at least one or more graph convolution layers connected to one or more convolution layers. The graph convolution block may be coupled to a spatial attention module.

To aggregate the multi-scale features, each decoder block 120′ may be configured to (i) upsample features from a previous decoder block with the features from a skip connection 116 connected to the corresponding encoder block 111a-111d to generate combined upsampled features 117 and (ii) direct the combined upsamples features to the decoding blocks, where each output of each stage of the decoding blocks are combined in a convolution layer (e.g., segmentation/prediction head).

Multi-Scale Hierarchical Cascaded Attention-based Decoder. In the example shown in FIG. 1C, the decoding blocks 113 (shown as 113a″, 113b″, 113c″, and 113d″), as a part of a multi-scale convolutional decoder, are configured with the multi-scale convolutional attention components (e.g., multi-scale convolutional attention module) employing at least one or more multi-scale convolution layers.

In some embodiments, the multi-scale convolutional attention block 120 (shown as multi-scale convolutional attention blocks (MSCAM) 120″) includes the at least one or more multi-scale convolution layers connected to one or more convolution layers. The graph convolution block may be coupled to a spatial attention module.

To aggregate the multi-scale features, each decoder block 120′ may be configured to (i) upsample features from a previous decoder block with the features from a skip connection 116 connected via a group attention gate (e.g., LGAG) to the corresponding encoder block 111a-111d to generate combined upsampled features 117 and (ii) direct the combined upsamples features to the decoding blocks, where each output of each stage of the decoding blocks are combined in a convolution layer (e.g., segmentation/prediction head).

### Example Method

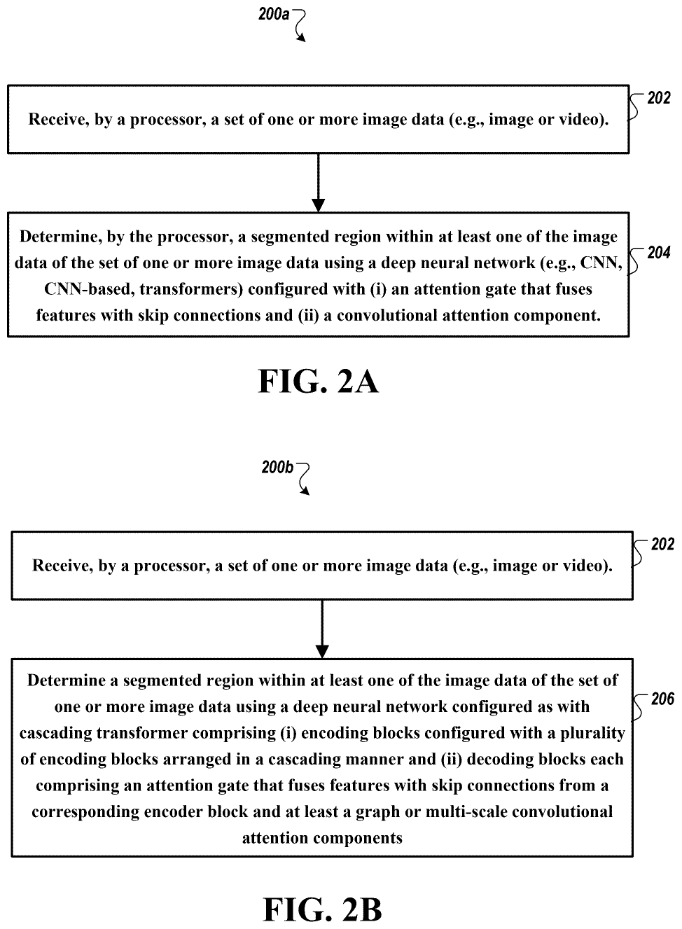

FIGS. 2A and 2B each shows an example operation flow 200 (shown as 200a, 200b) for the exemplary cascading attention decoding method. In FIG. 2A, the exemplary method 200a at step 202, includes receiving, by a processor, a set of one or more image data (e.g., image or video).

At step 204, the exemplary method 200a includes determining, by the processor, a segmented region within at least one of the image data of the set of one or more image data using a deep neural network (e.g., CNN, CNN-based, transformers) configured with (i) an attention gate that fuses features with skip connections and (ii) a convolutional attention component.

In FIG. 2B, the exemplary method 200b at step 202, includes receiving, by a processor, a set of one or more image data (e.g., image or video).

At step 206, the exemplary method 200b includes determining a segmented region within at least one of the image data of the set of one or more image data using a deep neural network configured as with cascading transformer comprising (i) encoding blocks configured with a plurality of encoding blocks arranged in a cascading manner and (ii) decoding blocks each comprising an attention gate that fuses features with skip connections from a corresponding encoder block and at least a graph or multi-scale convolutional attention components.

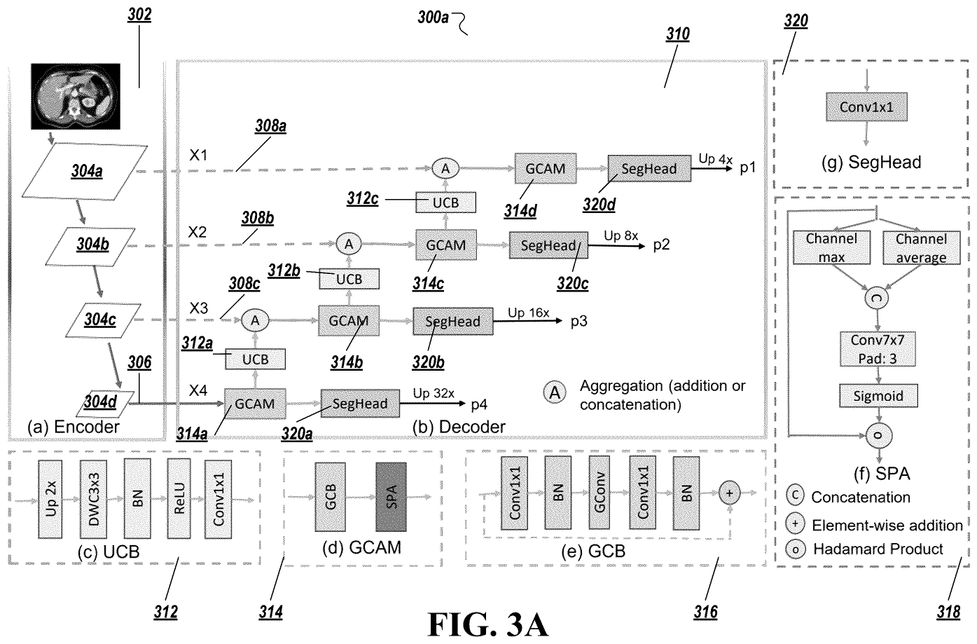

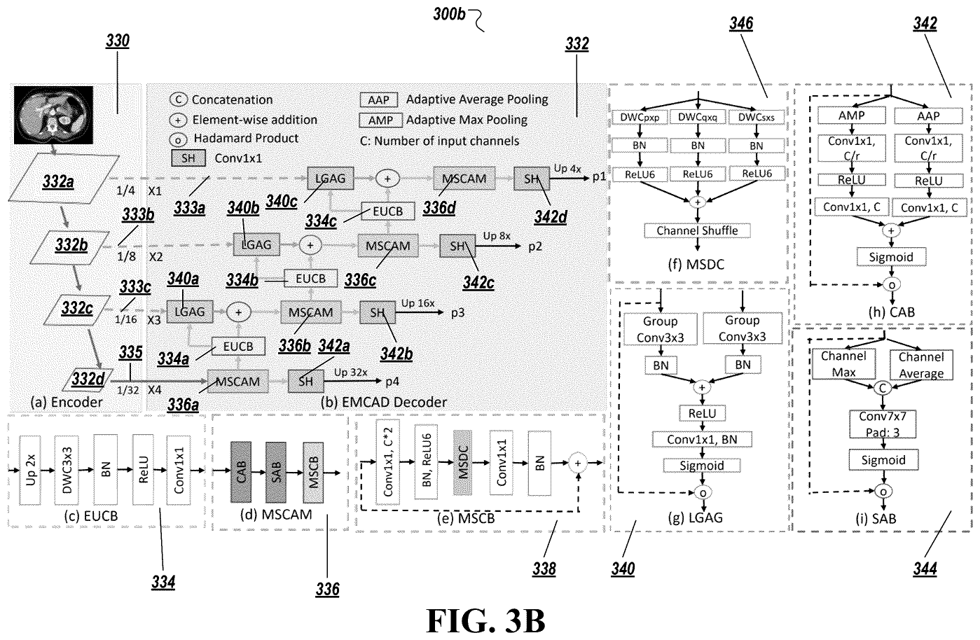

Further example details are provided herein in relation to the examples of FIGS. 3A and 3B.

### Example Cascaded Graph Convolutional Decoding Image Segmentation System

To ensure effective generalization and the ability to process multi-scale features in medical image segmentation, a cascaded graph convolutional (G-CASCADE) decoder of FIG. 1B can be formed by integrating two hierarchical backbone encoder networks such as pyramid vision transform (e.g., PVTv2) or multi-scale hierarchical vision transformer (MERIT) [29]. Other encoders may be used as described or referenced herein.

FIG. 3A shows an example impelmentation of a cascaded graph convolutional decoding network architecture 300a for the exemplary system of FIG. 1B in accordance with an illustrative embodiment. In FIG. 3A, a pyramid vision transform (e.g., PVTv2) encoder 302 is utilized for convolution operations embedding modules in four stages (e.g., 304a-304d; previously shown as 111) to consistently capture spatial information.

By utilizing the PVTv2-b2 encoder 302 (shown in FIG. 3A, subpanel a), the PVT-GCASCADE network architecture 300a can be created for the exemplary system. In the architecture 300a, the encoder 304 (previously shown as encoder blocks 111) can extract the features (X1, X2, X3, and X4) from 4 layers and feed them (i.e., X4 in the upsample path 306 and X3, X2, X1 in the skip connections 308a-308c) into a cascaded graph convolutional (G-CASCADE) decoder 310 (previously shown as 112′) as shown in FIG. 3A, subpanels (a) and (b). Then, the G-CASCADE decoder 310 can process them and produce prediction maps (e.g., prediction maps p₁-p₄) that correspond to the stages (e.g., 304a-304d) of the encoder network.

Cascaded graph convolutional decoder. State-of-the-art transformer-based models have limited (local) contextual information processing ability among pixels, and they may face difficulties in locating the more discriminating local features. Previous studies [9], [28], addressed the issue utilizing computationally expensive two-dimensional (2D) convolution blocks in the decoder. Although the convolution block can help to incorporate the local information, it can result in long-range attention deficits. In contrast, the exemplary system (e.g., FIG. 1B) may employ the cascaded graph convolutional (G-CASCADE) decoder 310 for its pyramid encoders.

As shown in FIG. 3A, subpanel (b), the G-CASCADE decoder 310 of the system 300a can consist of efficient up-convolution blocks 312 (UCBs, shown as 312a-c) to upsample the features, graph convolutional attention modules 314 (GCAMs, shown as 314a-314d) to robustly enhance the feature maps, and segmentation heads 320 (SegHeads, shown as 320a-320d) to get the segmentation output.

The decoder 310 can have any number of GCAMs (e.g., 314a-314d) corresponding to the stages (e.g., 304a-304d) of pyramid features from the encoder. In some embodiments, the number of stages of the decoder 310 is fewer than that of the encoder 304. In some embodiments, the number of stages of the decoder 310 is different than that of the encoder 304.

To aggregate the multi-scale features, the decoder 310 can first aggregate (e.g., addition or concatenation) the upsampled features (e.g., X4) from the previous decoder block with the features from the skip connections (e.g., X2, X3, X4). Afterward, the decoder 310 can process the concatenated features using the GCAM (e.g., 314a-314d) for enhancing semantic information. The decoder 310 can then send the output from each GCAM (e.g., 314a-314d) to a prediction head (segmentation head, e.g., 320a-320d). Finally, the decoder 310 can aggregate 4 different prediction maps (e.g., p₁-p₄) to produce the final segmentation output.

Graph convolutional attention module (GCAM) 314. The decoder 310 of the system 300a can use the graph convolutional attention modules (GCAMs, 314a-314d) to refine the feature maps. As shown in subpanel (d), GCAM can consist of a graph convolution block (GCB(.)) (e.g., 316) to refine the features preserving long-range attention and a spatial attention (SPA(.)) block (e.g., 318) [5] to capture the local contextual information as in Equation 1.

$$GCAM ( x ) = SPA ( GCB ( x ) ) \qquad ( Eq . l )$$

In Equation 1, x is the input tensor, and GCAM(.) represents the convolutional attention module. Due to using graph convolution, the GCAM can be more efficient than the convolutional attention module (CAM) developed in [28].

The graph convolution block 316 (GCB), shown in subpanel (e), can be used to enhance the features generated using cascaded expanding path. The GCB 316 can have the graphic design of a vision graph neural network (GNN) [13], consisting of a graph convolution layer GConv(.) having two 1×1 convolution layers C(.) each followed by a batch normalization layer BN(.), and a ReLU activation layer R(.). The graph convolution block 316 GCB(.) can be formulated as Equation

$$GCB ( x ) = R ( BN ( C ( GConv ( R ( BN ( C ( x ) ) ) ) ) ) ) \qquad ( Eq . 2 )$$

The GConv(.) in Equation 2 can be formulated using Equation 3.

$$GConv ( x ) = GELU ( BN ( D ynConv ( x ) ) ) \qquad ( Eq . 3 )$$

In Equation 3, DynConv(.) is a graph convolution (e.g., maxrelative, edge, GraphSAGE, and GIN) in dense dilated K-nearest neighbor (KNN) graph, and BN(.) and GELU(.) are batch normalization and Gaussian error linear unit (GELU) activation, respectively.

The SPA 318 shown in subpanel (f) may determine where to focus in a feature map, and then it can enhance those features. The spatial attention can be formulated as Equation 4.

$$SPA ( x ) = Sigmoid ( Conv ( [ {C}_{max} ( x ) , {C}_{avg} ( x ) ] ) ) ⊙ * x \qquad ( Eq . 4 )$$

In Equation 4, Sigmoid(.) is a Sigmoid activation function, Cₘₐₓ(.) and C_{avg}(.) represent the maximum and average values obtained along the channel dimension, respectively, Conv(.) is a 7×7 convolution layer with padding 3 to enhance local contextual information (as in [9]), and [symbol] is the Hadamard product.

Up-convolution block (UCB). UCB 312, shown in subpanel (c), can progressively upsample the features of the current layer to match the dimension to the next skip connection. Each UCB 312 layer can consist of an UpSampling Up(.) with scale-factor 2, a 3×3 depth-wise convolution DWC(.) with groups equal input channels, a batch normalization BN(.), a ReLU(.) activation, and a 1×1 convolution Conv(.). The UCB(.) can be defined as Equation 5.

$$UCB ( x ) = Conv ( ReLU ( BN ( DWC ( Up ( x ) ) ) ) ) \qquad ( Eq . 5 )$$

The UCB 312 employed by the system 300a can be light-weight as its 3×3 convolution can be replaced with a depth-wise convolution after upsampling.

Segmentation head (SegHead). SegHead 320, shown in subpanel (g), can take refined feature maps from the 4 stages of the decoder 310 as input and predict 4 output segmentation maps (e.g., p₁-p₄). Each SegHead layer 320 can consist of a 1×1 convolution Conviₓ₁(.), which can take feature maps having Ni channels (Ni is the number of channels in the feature map of stage i) as input and output with channels equal to the number of target classes for multi-class but 1 channel for binary prediction. The SegHead(.) can be formulated as Equation 6.

$$SegHead ( x ) = {Conv}_{1 \times 1} ( x ) \qquad ( Eq . 6 )$$

Multi-stage outputs and loss aggregation. As shown in subpanel (b), the 4 prediction heads (i.e., segmentation heads, 320a-320d) can generate 4 output segmentation maps p₁, p₂, p₃, and p₄ for the 4 stages of our G-CASCADE decoder 310.

The final segmentation output can be computed using additive aggregation as in Equation 7.

$$seg\_output = α {p}_{1} + β {p}_{2} + γ {p}_{3} + ζ {p}_{4} \qquad ( Eq . 7 )$$

In Equation 7, α, β, γ, and ξ are the weights of each prediction head (i.e., segmentation head), all of which can be set to 1.0 in the exemplary system. The final prediction output can be generated by applying the Sigmoid activation for binary segmentation and Softmax activation for multi-class segmentation, as shown in subpanel (f).

Following MERIT [29], the exemplary system can employ the combinatorial loss aggregation strategy (e.g., multi-stage feature-mixing loss aggregation MUTATION). Therefore, the loss for 2ⁿ-1 combinatorial predictions synthesized from n heads separately can be computed and summed up together. The additive combinatorial loss can be optimized during training.

Discussion MERIT can utilize 2 Max ViT encoders with varying window sizes for self-attention, thus enabling the capture of multi-scale features. MERIT's decoders may be improved, as described herein, with the G-CASCADE decoders, and MERIT's hybrid convolutional neural network-transformer (CNN-transformer) Max ViT encoder networks can be retained. In the MERIT-GCASCADE architecture, the first encoder can extract hierarchical feature maps from its 4 stages and then feed them to the first G-CASCADE decoder. Afterwards, the feedback from the final stage of the first G-CASCADE decoder can be aggregated to the input image and fed to the second encoder having different window sizes for self-attention.

The second encoder may extract feature maps from its 4 stages and feed them to the second G-CASCADE decoder. Cascaded skip connections like MERIT can be sent to the second G-CASCADE decoder, wherein 4 output segmentation maps may be generated from the 4 stages of the G-CASCADE second decoder. Finally, the segmentation maps from the 2 G-CASCADE decoders for 4 stages may be aggregated separately to produce 4 output segmentation maps. The G-CASCADE decoder may be adaptable and integrate-able with other hierarchical backbone networks.

### Multi-Scale Convolutional Attention Decoding Image Segmentation System

FIG. 3B shows an example efficient multi-scale convolutional attention decoding (EMCAD) system 300b configured to process the multi-stage features extracted from pretrained hierarchical vision encoders (e.g., 330) (e.g., 110) for high-resolution semantic segmentation. As shown in FIG. 3B, subpanel (b), EMCAD decoder 332 (previously shown as 112″) of the system 300b may consist of multiscale convolutional attention modules 336 (MSCAMs, shown as 336a-336d) to enhance the feature maps, large-kernel grouped attention gates 340 (LGAGs, shown as 340a-340c) to refine feature maps (e.g., X2-X4) fusing with the skip connection (e.g., 333a-333c) via gated attention mechanism, efficient up-convolution blocks 334 (EUCBs, shown as 334a-334c) for up-sampling followed by enhancement of feature maps, and segmentation heads (SHs, shown as 342a-342d) to produce the segmentation outputs (e.g., p₁-p₄).

More specifically, the system 300b may use 4 MSCAMs (e.g., 336a-336d) to refine pyramid features (i.e., X1, X2, X3, X4) extracted from the 4 stages (e.g., 332a-332d) of the encoder 332. After each MSCAM, the system 300b may use an SH to produce a segmentation map of that stage. Subsequently, the system 300b may upscale the refined feature maps using EUCBs (e.g., 334a-334c) and add them to the outputs from the corresponding LGAGs (e.g., 340a-340c). Finally, the system 300b may add 4 different segmentation maps to produce the final segmentation output.

Large-kernel grouped attention gate (LGAG). The system 300b may utilize a large-kernel grouped attention gate 340 (LGAG), shown in subpanel (g), to progressively combine feature maps (e.g., X1-X4) with attention coefficients, which may be learned by the network to allow higher activation of relevant features and suppression of irrelevant ones. This process employs a gating signal derived from higher-level features to control the flow of information across different stages of the network, thus enhancing its precision for medical image segmentation.

Unlike Attention UNet [41′], which uses 1×1 convolution to process gating signal g (features from skip connections) and input feature map x (upsampled features), in the LGAG qₐₜₜ(.) function, the LGAG 340 (shown in subpanel g) may process g and x by applying separate 3×3 group convolutions GC_{g}(.) and GCₓ(.), respectively. These convolved features may then be normalized using batch normalization (BN(.)) [27′] and merged through elementwise addition. The resultant feature map may be activated through a ReLU (R(.)) layer [39′].

Afterward, the LGAG 340 may apply a 1×1 convolution (C(.)) followed by BN(.) layer to get a single-channel feature map. The LGAG 340 may then pass the resultant single-channel feature map through a Sigmoid (σ(.)) activation function to yield the attention coefficients. The output of this transformation may be used to scale the input feature x through elementwise multiplication, producing the attention-gated feature LGAG (g, x).

The LGAG(.) 340 (shown in subpanel g) may be formulated as in Equations 8 and 9.

$${q}_{att} ( g , x ) = R ( BN ( {GC}_{g} ( g ) + BN ( {GC}_{x} ( x ) ) ) ) \qquad ( Eq . 8 )$$

$$LGAG ( g , x ) = x σ ( BN ( C ( {q}_{att} ( g , x ) ) ) ) \qquad ( Eq . 9 )$$

Due to using 3×3 kernel group convolutions in que(.), the LGAG 340 may capture comparatively larger spatial contexts with less computational cost.

Multi-scale convolutional attention module (MSCAM) 336. The system 300b may employ a multi-scale convolutional attention module (MSCAM, shown as 336a-336d) to refine the feature maps (e.g., X1-X4). MSCAM 336, as shown in subpanel (d), may consist of a channel attention block (CAB(.)) (e.g., 342) to put emphasis on pertinent channels, a spatial attention block [9′] (SAB(.)) (e.g., 344) to capture the local contextual information, and a multi-scale convolution block (MSCB(.)) (e.g., 338) to enhance the feature maps preserving contextual relationships. The MSCAM(.) 336 (shown in subpanel d) may be defined in Equation 10.

$$MSCAM ( x ) = MSCB ( SAB ( CAB ( x ) ) ) \qquad ( Eq . 10 )$$

In Equation 10, x is the input tensor. Due to using depth-wise convolution in multiple scales, the MSCAM 336 may be more effective with lower computational cost than the convolutional attention module (CAM) proposed in [42′].

Multi-scale convolution block 338 (MSCB) (shown in subpanel e), employed by the MSCAM 336, may enhance the features (e.g., X4) generated by a cascaded expanding path (e.g. 335). The MSCB 338 may follow the design of the inverted residual block (IRB) of MobileNetV2 [45′]. However, unlike IRB, the MSCB 338 may perform depth-wise convolution at multiple scales and use channel shuffle [60′] to shuffle channels across groups.

More specifically, in the MSCB 338, the number of channels may first be expanded (i.e., expansion factor=2) using a point-wise (1×1) convolution layers PWC₁(.) followed by a batch normalization layer BN(.) and a ReLU6 [31′] activation layer R6(.). Then, a multi-scale depth-wise convolution MSDC(.) (e.g., 346) may be used to capture both multi-scale and multiresolution contexts. As depth-wise convolution overlooks the relationships among channels, a channel shuffle operation may be used to incorporate relationships among channels. Afterward, another point-wise convolution PWC₂(.) followed by a BN(.) may be used to transform back the original number of channels, which can also decode dependency among channels. The MSCB(.) (e.g., 338, shown in FIG. 3B, subpanel e) may be formulated as shown in Equation 11.

$$MSCB ( x ) = BN ( {PWC}_{2} ( CS ( MSDC ( R 6 ( BN ( {PWC}_{1} ( x ) ) ) ) ) ) ) \qquad ( Eq . 11 )$$

In Equation 11, parallel MSDC(.) 346 (shown in subpanel f) for different kernel sizes (KS) may be formulated using Equation 12.

$$MSDC ( x ) = {\sum }_{ks ∈ KS} {DWCB}_{ks} ( x ) , \\ where {DCWCB}_{ks} ( x ) = R 6 ( BN ( {DWC}_{ks} ( x ) ) ) \qquad ( Eq . 12 )$$

In Equation 12, DWCₖₛ(.) is a depth-wise convolution with the kernel size ks, and BN(.) and R6(.) are batch normalization and ReLU6 activation, respectively. Additionally, the MSDC(.) may use the recursively updated input x, where the input x is residually connected to the previous DWCBₖₛ(.) for better regularization, as shown in Equation 13.

$$x = x + {DWCB}_{ks} ( x ) \qquad ( Eq . 13 )$$

Channel attention block (CAB) (e.g., 342) may assign different levels of importance to each channel, thus emphasizing more relevant features while suppressing less useful ones. The CAB 342 may determine which feature maps to focus on (and then refine them).

Following [57′], in the CAB 342 shown in subpanel (h), the adaptive maximum pooling (Pₘ(.)) and adaptive average pooling (Pₐ(.)) may be applied to the spatial dimensions (i.e., height and width) to extract the most significant feature of the entire feature map per channel. Then, for each pooled feature map, the number of channels may be reduced r=1/16 times separately using a point-wise convolution (C₁(.)) followed by a ReLU activation (R). Afterward, the original channels may be recovered using another point-wise convolution (C₂(.)). Then, both recovered feature maps may be added, and Sigmoid (o) activation may be applied to estimate attention weights. Finally, these weights may be incorporated to input x using the Hadamard product ( [symbol] ) The CAB(.) 342 (shown in subpanel h) may be defined using Equation 14.

$$CAB ( x ) = σ ( {C}_{2} ( R ( {C}_{1} ( {P}_{m} ( x ) ) ) ) + {C}_{2} ( R ( {C}_{1} ( {P}_{a} ( x ) ) ) ) ) x \qquad ( Eq . 14 )$$

Spatial attention block 344 (SAB), shown in FIG. 3B, subpanel (i), may be used to mimic the attentional processes of the human brain by focusing on specific parts of an input image. The SAB 344 may determine where to focus in a feature map, and then it may enhance those features. This process may enhance the model's ability to recognize and respond to relevant spatial features, which may be crucial for image segmentation, where the context and location of objects may influence the output.

In the SAB 344, channel maximum (Chₘᵤᵣ(.)) and average (Ch_{arg}(.)) values may be pooled along the channel dimension to pay attention to local features. Then, a large kernel (i.e., 7×7 as in [17′]) convolution layer may be used to enhance local contextual relationships among features. Afterward, the Sigmoid activation (σ) may be applied to calculate attention weights. Finally, these weights may be fed to the input x using Hadamard product ( [symbol] ) to attend information in a more targeted way. The SAB(.) 344 (shown in subpanel i) may be defined using Equation 15.

$$SAB ( x ) = σ ( LKC ( [ {Ch}_{max} ( x ) , {Ch}_{avg} ( x ) ] ) ) x \qquad ( Eq . 15 )$$

Efficient up-convolution block (EUCB) 334. The system 300b may use an efficient up-convolution block 334 (EUCB) to progressively upsample the feature maps of the current stage to match the dimension and resolution of the feature maps from the next skip connection. The EUCB 334 can first use an UpSampling operation Up(.) with scale-factor 2 to upscale the feature maps. Then, the EUCB 334 may enhance the upscaled feature maps by applying a 3×3 depth-wise convolution DWC(.) followed by a BN(.) and a ReLU(.) activation. Finally, a 1×1 convolution C_{1×1}(.) may be used to reduce the number of channels to match with the next stage. The EUCB(.) 334 (shown in subpanel c) may be formulated as in Equation 16.

$$EUCB ( x ) = {C}_{1 \times 1} ( ReLU ( BN ( DWC ( Up ( x ) ) ) ) ) \qquad ( Eq . 16 )$$

Due to using depth-wise convolution instead of 3×3 convolution, the EUCB 334 may be very efficient.

Segmentation head (SH) 342. The system 300b may use segmentation heads (e.g., 342a-342d) to produce the segmentation outputs from the refined feature maps of the 4 stages of the decoder. The SH layer may apply a 1×1 convolution Conviₓ₁(.) to the refined feature maps having chᵢ channels (chᵢ is the number of channels in the feature map of stage i) and produces output with a number of channels equal to the number of classes in target dataset for multi-class but 1 channel for binary segmentation. The SH(.) may be formulated as shown in Equation 17.

$$SH ( x ) = {Conv}_{1 \times 1} ( x ) \qquad ( Eq . 17 )$$

Multi-stage loss and outputs aggregation. The exemplary EMCAD decoder's 4 segmentation heads may produce 4 prediction maps p₁, p₂, p₃, and p₄. across its stages.

The loss aggregation of the predictions may be computed using a combinatorial approach to loss combination called MUTATION, inspired by the work of MERIT [43′] for multi-class segmentation. This may involve calculating the loss for all possible combinations of predictions derived from 4 heads, totaling 2⁴−1=15 unique predictions, and then summing these losses. The system 300b may focus on minimizing this cumulative combinatorial loss during the training process. For binary segmentation, the additive loss (i.e., aggregated loss) like [42′] with an additional term Lₚ_{₁}₊ₚ_{₂}₊ₚ_{₃}₊ₚ_{₄} may be optimized as in Equation 18.

$${L}_{total} = α {L}_{{p}_{1}} + β {L}_{{p}_{2}} + γ {L}_{{p}_{3}} + ζ {L}_{{p}_{4}} + δ {L}_{{p}_{1} + {p}_{2} + {p}_{3} + {p}_{4}} \qquad ( Eq . 18 )$$

In Equation 18, Lₚ_{₁}, Lₚ_{₂}, Lₚ_{₃}, and Lₚ_{₄}, are the losses of each individual prediction maps. α=β=γ=ξ=δ=1.0 may be the weights assigned to each loss.

The prediction map, p₄, from the last stage of the EMCAD decoder 332 may be considered as the final segmentation map. Then, the final segmentation output may be obtained by employing a Sigmoid function for binary or a Softmax function for multi-class segmentation.

Alternative architectures. To show the generalization, effectiveness, and ability to process multi-scale features for medical image segmentation, the system 300b may employ tiny (PVTv2-B0) and standard (PVTv2-B2) encoder networks of PVTv2 [56′]. However, the EMCAD decoder 332 in the exemplary system may be adaptable and seamlessly compatible with other hierarchical backbone networks.

PVTv2 differs from conventional transformer patch embedding modules by applying convolutional operations for consistent spatial information capture. Using PVTv2-b0 (Tiny) and PVTv2-b2 (Standard) encoders [56′], the PVT-EMCAD-B0 and PVT-EMCAD-B2 architectures (i.e., EMCAD-based systems) may be developed.

To adopt PVTv2, the system 300b may first extract the features (X1, X2, X3, and X4) from 4 layers and feed them (i.e., X4 in the upsample path 335 and X3, X2, X1 in the skip connections 333a-333c) into the EMCAD decoder 332 as shown in subpanels (a) and (b). The EMCAD decoder 332 may then process them and produce 4 segmentation maps (e.g., p₁-p₄) that correspond to the 4 stages (e.g., 332a-332d) of the encoder network 330.

### Multi-Scale Hierarchical Vision Transformer Image Segmentation System

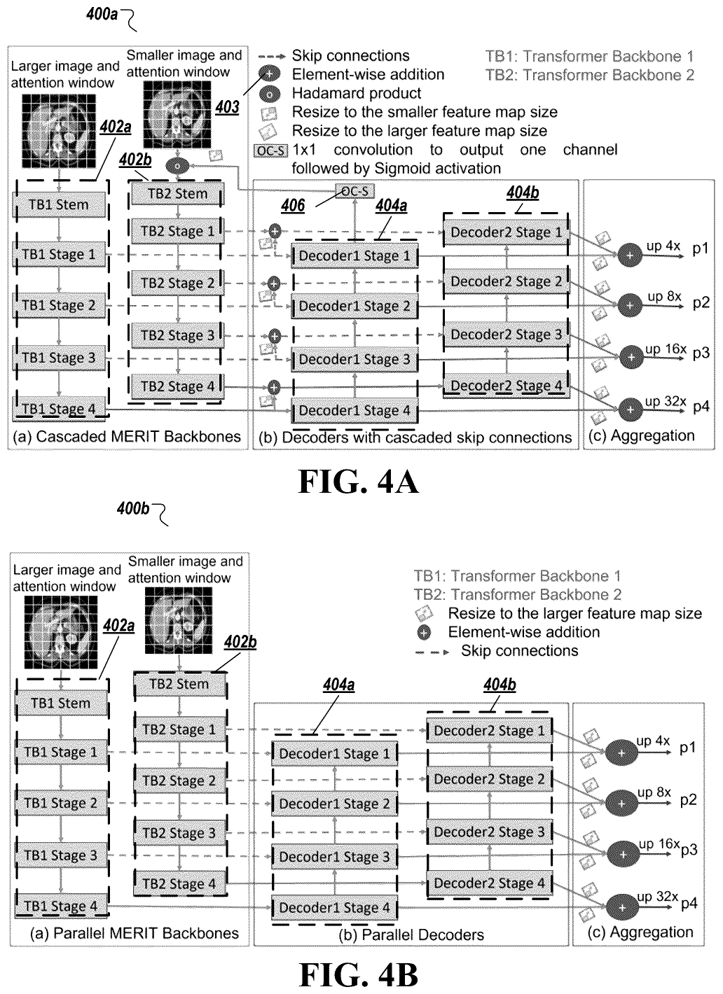

FIGS. 4A-4B each shows an example multi-scale hierarchical vision transformer (MERIT) system. FIG. 4A shows an example MERIT system 400a configured with a cascaded MERIT backbone. FIG. 4B shows an example MERIT system 400b configured with a parallel MERIT backbone.

Pure transformers have limited (spatial) contextual information processing ability among pixels. As a result, the transformer-based models face difficulties in locating discriminative local features. To address this issue, the MERIT system (e.g. 400a, 400b) employs an attention-based cascaded decoder, CASCADE [78′] (Decoder1, shown as 404a, and Decoder2, shown as 404b), for multi-stage feature refinement and aggregation. CASCADE decoder (e.g., 404a, 404b) may use the attention gate (AG) [77′] for cascaded feature aggregation and the convolutional attention module (CAM) for robust feature map enhancement. CASCADE decoder has 4 CAM blocks for the 4 stages (e.g., Stage 1-Stage 4) of hierarchical features from the transformer backbone (e.g., TB1, TB2) and 3 AGs for 3 skip connections. CASCADE decoder may aggregate the multi-resolution features by combining the upsampled features from the previous stage of the decoder with the features from the skip connections using AG. Then, the CASCADE decoder may process the aggregated features using the CAM module (consists of channel attention [71′] followed by spatial attention [65′], which may group pixels together and suppress background information. Lastly, the CASCADE decoder may send the output from the CAM block of each stage to a prediction head to produce prediction maps (e.g., p₁, p₂, p₃, p₄).

The MERIT system (e.g. 400a, 400b) may produce prediction maps from the 4 stages of the CASCADE decoder. The MERIT system (e.g., 400a, 400b) may aggregate (add) the prediction maps for each stage of the 2 decoders and generate the final prediction map ŷ using Equation 19.

$$\hat{y} = α \times {p}_{1} + β \times {p}_{2} + γ \times {p}_{3} + ψ \times {p}_{4} \qquad ( Eq . 19 )$$

In Equation 19, p₁, p₂, p₃, and p₄ represent the prediction maps, and α, β, γ, and Ψ are the weights of each prediction head. The MERIT system (e.g., 400a, 400b) may use the value of 1.0 for α, β, γ, and Ψ. Finally, the MERIT system (e.g., 400a, 400b) may apply Softmax activation on ŷ to get the multi-class segmentation output.

The MERIT system (e.g., 400a, 400b) may employ a SoA transformer, Max ViT [81′]. Specifically, the MERIT system (e.g., 400a, 400b) may use 2 instances of Max ViT-S (standard) backbone with 8×8 and 7×7 attention windows as its MERIT backbone (e.g., TB1, TB2). Each Max ViT backbone may have 2 Stem blocks (e.g., TB1 Stem, TB2 Stem) followed by 4 stages (e.g., TB1 Stage 1-4, TB2 Stage 1-4) that may consist of multiple (i.e., 2, 2, 5, 2) Max ViT blocks. Each Max ViT block may be built with a Mobile Convolution Block (MBConv), a Block Attention having Block Self-Attention (SA) followed by a Feed Forward Network (FFN), a Grid Attention having a Grid SA followed by an FFN. Additionally, other transformer backbones may also be used with the MERIT system (e.g., 400a, 400b).

Cascaded MERIT. In the cascaded MERIT system 400a, feedback from a backbone (e.g., 402a) may be added (i.e., cascaded 403) to the next backbone (e.g., 402b). Specifically, the hierarchical features from 4 different stages (e.g., TB2 Stage 1-4) of the backbone network (e.g., 402b) may be extracted and cascaded with the features from the previous backbone (e.g., 402a), and then pass to the skip connections and bottleneck modules of the respective decoders (e.g., 404a, 404b), except the first decoder. The feedback from the decoder of one backbone (e.g., 404a) may also be passed to the next backbone (e.g., 402b), except the last (e.g., 404b). This design may capture the multi-scale, as well as multi-resolution features due to using multiple attention windows and hierarchical features. It also refines the features well by adding some feedback from the decoder of a backbone to the next backbone via cascaded skip connections.

In FIG. 4A, subpanel (a) presents the Cascaded MERIT architecture with 2 backbone networks (e.g., 402a, 402b). For each backbone network, the images with size (H, W) are first put into a Stem layer (e.g., TB1 Stem, TB2 Stem), which may reduce the resolution of the features to (H/4, W/4). Afterward, these features may be passed through 4 stages of transformer backbones (e.g., TB1 Stage 1-4, TB2 Stage 1-4), which may reduce the resolution of the features by 2 times at each stage, except the fourth. The features from the last stage of the first decoder 404a may be combined with the input image to cascade it with the second backbone 402b To do this, the number of channels may be reduced to one, and logits may be produced by applying a 1×1 convolution followed by Sigmoid activation (shown as 406). The feature map may also be resized to the input resolution (i.e., 224×224) of Backbone 2 (i.e., 402b). In some embodiments, features of MERIT may be implemented with configurations of FIGS. 1B and 1C.

Parallel MERIT. Unlike Cascaded MERIT in system 400a, in the MERIT backbone of the system 400b, input images of multiple resolutions in parallel may be passed into separate hierarchical transformer backbone encoders (e.g., 402a, 402b) with different attention windows. In other words, there is no cascading operations 403 and 406 between the transformer backbones 402a and 402b in system 400b.

Similar to the Cascaded MERIT, the hierarchical features from 4 different stages (e.g., TB1 Stage 1-4, TB2 Stage 1-4) of the backbone networks (e.g., 402a, 402b) can be extracted and passed to the respective parallel decoders (e.g., 404a, 404b). The system 400b may also capture multi-scale features due to using hierarchical backbones with multiple attention windows.

In the system 400b, input images may be passed through similar steps in the backbone networks, just as in the system 400a. However, unlike system 400a, system 400b may only share information among the backbone networks at the very end during the feature aggregation step (FIG. 4B, subpanel c).

Decoder. Each transformer backbone (e.g., 402a, 402b) may employ a separate decoder. As shown in FIG. 4A, subpanel (b), cascaded skip connections may be used in the decoder of the cascaded MERIT system 400a. The skip connections from the first backbone may be added to the skip connections of the second backbone network. In this case, information may be shared across backbones in 3 phases, e.g., during backbone cascading, skip connections cascading, and aggregating prediction maps. This sharing of information may capture richer information than the single-resolution backbone, as well as the parallel MERIT system 400b.

Unlike system 400a, in system 400b, the parallel backbones may have 2 parallel decoders. Each decoder may have 4 stages that correspond to 4 stages of the transformer backbone. The multi-stage prediction maps produced by the decoders in system 400b may be aggregated at the aggregation step shown in FIG. 4B, subpanel (c).

Multi-stage feature-mixing loss aggregation (MUTATION). Multi-stage feature mixing loss aggregation method for image segmentationcan enable better model training. In MUTATION method, prediction maps may be created by combining the available prediction maps. So, all the prediction maps from different stages of a network may be taken as input, and the losses of prediction maps generated may be aggregated using 2ⁿ-1 non-empty subsets of n prediction maps. For example, if a network produces 4 prediction maps, the multi-stage feature-mixing loss aggregation method may produce a total of 15 (i.e., 2⁴-1) prediction maps, including 4 original maps.

This mixing method is simple, as it may not require additional parameters to calculate, and it may not introduce inference overheads. Due to its potential benefits, this method may be used with any multi-stage image segmentation or dense prediction networks.

### Vision Encoders and Medical Image Segmentation

Vision Encoders. Convolutional Neural Networks (CNNs) [21′-23′], [32′], [35′], [45′-48′] may be foundational as encoders due to their proficiency in handling spatial relationships in images. More precisely, AlexNet [32′] and VGG [46′] pave the way, leveraging deep layers of convolutions to extract features progressively. GoogleNet [47′] introduces the inception module, allowing more efficient computation of representations across various scales. ResNet [21′] introduces residual connections, enabling the training of networks with substantially more layers by addressing the vanishing gradients problem. MobileNets [22′], [45′] bring CNNs to mobile devices through lightweight, depth-wise separable convolutions. EfficientNet [48′] introduces a scalable architectural design to CNNs with compound scaling. Although CNNs may be pivotal for many vision applications, they may lack the ability to capture long-range dependencies within images due to their inherent local receptive fields.

Vision Transformers (ViTs), pioneered by Dosovitskiy et al. [18′], may enable the learning of long-range relationships among pixels using Self-attention (SA). Since then, ViTs have been enhanced by integrating CNN features [49′], [56′], developing self-attention (SA) blocks [34′], [49′], and introducing architectural designs [55′], [58′]. The Swin Transformer [34′] may incorporate a sliding window attention mechanism, while SegFormer [58′] may provide Mix-FFN blocks for hierarchical structures. PVT uses spatial reduction attention, refined in PVTv2 [56′] with overlapping patch embedding and a linear complexity attention layer. Max ViT [49′] introduces a multi-axis self-attention to form a hierarchical CNN-transformer encoder. Although ViTs may address the CNN's limitation in capturing long-range pixel dependencies [21′-23′], [32′], [35′], [45′], [46′], [47′], [48′], they may face challenges in capturing the local spatial relationships among pixels.

Medical image segmentation. Medical image segmentation may involve pixel-wise classification to identify various anatomical structures like lesions, tumors, or organs within different imaging modalities such as endoscopy, MRI, or CT scans [8′]. U-shaped networks [7′], [19′], [24′], [26′], [37′], [41′], [44′], [62′] may be favored due to their simple but effective encoder-decoder design. The UNet [44′] pioneered this approach with its use of skip connections to fuse features at different resolution stages. UNet++ [62′] may evolve this design by incorporating nested encoder-decoder pathways with dense skip connections. Expanding on these ideas, UNet 3+ [24′] introduces comprehensive skip pathways that facilitate full-scale feature integration. Further advancement comes with DC-UNet [37′], which may integrate a multi-resolution convolution scheme and residual paths into its skip connections. The DeepLab series, including DeepLabv3 [10′] and DeepLabv3+ [11′], introduce atrous convolutions and spatial pyramid pooling to handle multi-scale information. SegNet [2′] may use pooling indices to upsample feature maps, preserving the boundary details. nnU-Net [19′] can configure hyperparameters based on the specific dataset characteristics using standard 2D and 3D UNets. Collectively, these U-shaped models have become a benchmark for success in the domain of medical image segmentation.

Vision transformers have emerged as a formidable force in medical image segmentation, harnessing the ability to capture pixel relationships at global scales [5′], [8′], [17′], [42′], [43′], [52′], [58′], [61′]. TransUNet [8′] presents a blend of CNNs for local feature extraction and transformers for global context, enhancing both local and global feature capture. Swin-Unet [5′] may extend this by incorporating Swin Transformer blocks [34′] into a U-shaped model for both encoding and decoding processes. Building on these concepts, MERIT [43′] introduces a multi-scale hierarchical transformer, which employs SA across different window sizes, thus enhancing the model capacity to capture multiscale features critical for medical image segmentation.

The integration of attention mechanisms has been investigated within CNNs [20′], [41′] and transformer-based systems [17′] for enhancing medical image segmentation. PraNet [20′] employs a reverse attention strategy for feature refinement. PolypPVT [17′] leverages PVTv2 [56′] as its backbone encoder and incorporates CBAM [57′] within its decoding stages. The CASCADE [42′] presents a cascaded decoder, combining channel [23′] and spatial [9′] attention to refine features at multiple stages, extracted from a transformer encoder, culminating in high-resolution segmentation outputs. While CASCADE may achieve notable performance in segmenting medical images by integrating local and global insights from transformers, it may be computationally inefficient due to the use of triple 3×3 convolution layers at each decoder stage.

### Experimental Results and Additional Examples

A set of studies was conducted to develop the exemplary image segmentation systems and methods for resource-efficiently enhancing feature maps over long-range correlations among pixels in image segmentation processes. Experimental results, implementation details, and additional examples for each of the Embodiments #1-#3 are provided below.

### Embodiment #1—Cascaded Graph Convolutional Decoding Image Segmentation System (i.e., G-CASCADE-Based System, G-CASCADE Decoder)

The study implemented G-CASCADE-based systems (e.g., PVT-GCASCADE, MERIT-GCASCADE) and compared them against the state-of-the-art (SoA) methods when operating on different datasets. G-CASCADE enhances feature maps while preserving longrange information captured by transformers which is crucial for accurate medical image segmentation. Due to using graph convolution blocks instead of 3×3 convolution blocks, G-CASCADE is computationally very efficient.

Experimental results show that G-CASCADE outperforms a recent decoder, CASCADE, in DICE scores with 80.8% fewer parameters and 82.3% fewer FLOPs. The experimental results also demonstrate the superiority of the G-CASCADE decoder over SOTA methods on five public medical image segmentation benchmarks. The instant decoder may improve other downstream medical image segmentation and semantic segmentation tasks, e.g., reconstruction.

Datasets. The study trained the G-CASCADE-based systems (e.g., PVT-GCASCADE, MERIT-GCASCADE) on 5 types of datasets: the Synapse multi-organ dataset, the automated cardiac diagnosis challenge (ACDC) dataset, the ISIC2018 dataset, the polyp datasets, and the retinal vessels segmentation datasets.

The Synapse multi-organ dataset contained 30 abdominal computer tomographic (CT) scans, which had 3779 axial contrast-enhanced slices. Each CT scan had 85-198 slices of 512×512 pixels. Similar to TransUNet [4], the study divided the dataset randomly into 18 scans for training (2212 axial slices) and 12 scans for validation. The study segmented only 8 abdominal organs, i.e., the aorta, gallbladder (GB), left kidney (KL), right kidney (KR), liver, pancreas (PC), spleen (SP), and stomach (SM).

The ACDC dataset contained 100 cardiac MRI scans, each of which consisted of 3 organs: right ventricle (RV), myocardium (Myo), and left ventricle (LV). Following TransUNct [4], the study used 70 cases (1930 axial slices) for training, 10 for validation, and 20 for testing.

The ISIC2018 dataset was a skin lesion segmentation dataset [8], consisting of 2596 images with corresponding annotations. In the experiments, the study resized the images to 384×384 resolution. The study randomly split the images into 80% for training, 10% for validation, and 10% for testing.

Polyp dataset types may include different individual datasets (e.g., Kvasir, CVC-ClinicDB, EndoScene, ColonDB). Kvasir contained 1,000 polyp images collected from the polyp class in the Kvasir-SEG dataset [18]. CVC-ClinicDB [1] consisted of 612 images extracted from 31 colonoscopy videos. Following CASCADE [28], the study adopted the same 900 and 550 images from Kvasir and CVC-ClinicDB, respectively, as the training set. The study used the remaining 100 and 62 images as the respective testsets. To assess the generalizability of the G-CASCADE decoder, the study used 2 unseen test datasets, namely EndoScene [35], and ColonDB [32]. EndoScene and ColonDB consisted of 60 and 380 images, respectively.

Retinal vessels segmentation dataset type can include different individual datasets (e.g., DRIVE, CHASE_DB1). The DRIVE dataset had 40 retinal images with segmentation annotations. All the retinal images in this dataset were 8-bit color images of resolution 565×584 pixels. The official splits contained a training set of 20 images and a test set of 20 images. The CHASE_DB1 [3] dataset contained 28 color retina images of 999×960 pixels resolution. There were 2 manual annotations of each image for segmentation. The study used the first annotation as the ground truth. Following [22], the study used the first 20 images for training and the remaining 8 images for testing.

Evaluation metrics. The study used dice similarity coefficient (DICE), mean intersection overunion (mIoU), and 95% Hausdorff Distance (HD95) as evaluation metrics for performance on the Synapse multi-organ dataset. However, for the ACDC dataset, the study used only DICE score as an evaluation metric.

The study used DICE and mloU as the evaluation metrics for polyp segmentation and ISIC2018 datasets. The study used accuracy (Acc), sensitivity (Sen), specificity (Sp), DICE, and mloU scores as evaluation metrics. The study reported the percentage (%) score averaging over 5 runs for all datasets.

The DICE score (denoted as DSC (Y, Y)), mIoU score (denoted as loU (Y, Y)), and HD95 distance (denoted as DH (Y,Y)) was calculated using Equations 20, 21, and 22, respectively.

$$DSC ( Y , \hat{Y} ) = \frac{2 \times \vert Y ⋂ \hat{Y} \vert}{\vert Y \vert + \vert \hat{Y} \vert} \times 100 \qquad ( Eq . 20 )$$

$$IoU ( Y , \hat{Y} ) = \frac{\vert Y ⋂ \hat{Y} \vert}{\vert Y ⋃ \hat{Y} \vert} \times 100 \qquad ( Eq . 21 )$$

$${D}_{H} ( Y , \hat{Y} ) = max \{ {max}_{y ∈ Y} {min}_{\hat{y} ∈ \hat{Y}} d ( y , \hat{y} ) , \{ {max}_{\hat{y} ∈ \hat{Y}} {min}_{y ∈ Y} d ( y , \hat{y} ) \} \qquad ( Eq . 22 )$$

In Equations 20, 21, 22, Y and Ý are the ground truth and predicted segmentation map, respectively.

Implementation details. The study used Pytorch 1.11.0 to implement the G-CASCADE-based system, and conducted experiments. The study trained all models on a single NVIDIA RTX A6000 GPU with 48 GB of memory and used the PVTv2-b2 and Small CascadedMERIT as representative network. The study used the pre-trained weights on ImageNet for both PVT and MERIT backbone networks. The study trained the models using the AdamW optimizer with both a learning rate and weight decay of 0.0001.

To configure the graph convolution block (GCB), the study constructed a dense dilated graph using K=11 neighbors for KNN and used the Max-Relative (MR) graph convolution in all experiments. The batch normalization was used after MR graph convolution. Following ViG [13], the study also used the relative position vector for graph construction and reduction ratios of [1, 1, 4, 2] for graph convolution blocks in different stages.

For the Synapse multi-organ dataset, the study used a batch size of 6 and trained each model for a maximum of 300 epochs. The study used the input resolution of 224×224 for PVT-GCASCADE and (256×256, 224×224) for MERIT-GCASCADE. The study applied random rotation and flipping for data augmentation. The combined weighted Cross-entropy (0.3) and DICE (0.7) loss were utilized as the loss function.

For the ACDC dataset, the study trained each model for a maximum of 150 epochs with a batch size of 12. The study set the input resolution as 224×224 for PVT-GCASCADE and (256×256, 224×224) for MERIT-GCASCADE. The study applied random flipping and rotation for data augmentation. The study optimized the combined weighted Cross-entropy (0.3) and DICE (0.7) loss function.

For the ISIC2018 dataset, the study resized the images into 384×384 resolution. Then, the study trained our model for 200 epochs with a batch size of 4 and a gradient clip of 0.5. The study optimized the combined weighted BCE and weighted IoU loss function.

For polyp datasets, the study resized the image to 352×352 and used a multi-scale {0.75, 1.0, 1.25} training strategy with a gradient clip limit of 0.5 like CASCADE [28]. The study used a batch size of 4 and trained each model a maximum of 200 epochs. The study optimized the combined weighted BCE and weighted IoU loss function.

For each retinal vessel segmentation dataset, DRIVE and CHASE_DB1 [3], the study first extended the training set using horizontal flips, vertical flips, horizontal-vertical flips, random rotations, random colors, and random Gaussian blurs. Through this process, the study got 260 images, including the 20 original training images. The study used 26 of these images for validation that belonged to 4 randomly selected original images. In the case of the DRIVE dataset, the study resized the images into 768×768 resolution for PVT and (768×768, 672×672) resolutions for MERIT. In the case of CHASE_DB1, the study used 960×960 resolution inputs for PVT and (768×768, 672×672) resolution inputs for MERIT. However, the study resized the output segmentation maps to the original resolution to get evaluation metrics during inference. The study used random flips and rotations with a probability of 0.5 as augmentation methods during training. The study optimized the combined weighted binary cross entropy (BCE) and weighted mloU loss function. The MUTATION was used to aggregate multi-stage loss. The study trained the networks for 200 epochs with a batch size of 4 and 2 for DRIVE and CHASE_DB, respectively.

The study compared the G-CASCADE-based systems (e.g., PVT-GCASCADE and MERIT-GCASCADE) with state-of-the-art (SOA) CNN and transformer-based segmentation methods on Synapse multi-organ, ACDC, ISIC2018 [8], Polyp (i.e., Endoscene [35], CVC-ClinicDB [1], Kvasir [18], ColonDB [32]), and retinal vessel segmentation datasets.

Quantitative results on Synapse multi-organ dataset. Table 1 presents the performance of different CNN—and transformer-based methods on the Synapse multi-organ segmentation dataset. The study reported only DICE scores for individual organs. The study got the results of UNet, AttnUNet, PolypPVT, SSFormerPVT, TransUNet, and SwinUNet from [28]. The study reproduced the results of Cascaded MERIT with a batch size of 6. The study averaged G-CASCADE results over 5 runs. In Table 1, ↑(↓) denotes the higher (lower), the better, and the best results are shown in bold.

TABLE 1

| Architectures/Methods | DICE↑ | Average HD95↓ | mIoU↑ | Aorta | GB |
|---|---|---|---|---|---|
| UNet [30] | 70.11 | 44.69 | 59.39 | 84.00 | 56.70 |
| AttnUNet [27] | 71.70 | 34.47 | 61.38 | 82.61 | 61.94 |
| R50 + UNet [4] | 74.68 | 36.87 | — | 84.18 | 62.84 |
| R50 + AttnUNet [4] | 75.57 | 36.97 | — | 55.92 | 63.91 |
| SSFormerPVT [38] | 78.01 | 25.72 | 67.23 | 82.78 | 63.74 |
| PolypPVT [9] | 78.08 | 25.61 | 67.43 | 82.34 | 66.14 |
| TransUNet [4] | 77.61 | 26.9 | 67.32 | 86.56 | 60.43 |
| SwinUNet [2] | 77.58 | 27.32 | 66.88 | 81.76 | 65.95 |
| MT-UNet [37] | 78.59 | 26.59 | — | 87.92 | 64.99 |
| MISSFormer [16] | 81.96 | 18.20 | — | 86.99 | 68.65 |
| PVT-CASCADE [28] | 81.06 | 20.23 | 70.88 | 83.01 | 70.59 |
| TransCASCADE [28] | 82.68 | 17.34 | 73.48 | 86.63 | 68.48 |
| Cascaded MERIT [29] | 84.32 | 14.27 | 75.44 | 86.67 | 72.63 |
| PVT-GCASCADE | 83.28 | 15.83 | 73.91 | 86.50 | 71.71 |
| (This study) | | | | | |
| MERIT-GCASCADE | 84.54 | 10.38 | 75.83 | 88.05 | 74.81 |
| (This study) | | | | | |

| Architectures/Methods | KL | KR | Liver | PC | SP | SM |
|---|---|---|---|---|---|---|
| UNet [30] | 72.41 | 62.64 | 86.98 | 48.73 | 81.48 | 67.96 |
| AttnUNet [27] | 76.07 | 70.42 | 87.54 | 46.70 | 80.67 | 67.66 |
| R50 + UNet [4] | 79.19 | 71.29 | 93.35 | 48.23 | 84.41 | 73.92 |
| R50 + AttnUNet [4] | 79.20 | 72.71 | 93.56 | 49.37 | 87.19 | 74.95 |
| SSFormerPVT [38] | 80.72 | 78.11 | 93.53 | 61.53 | 87.07 | 76.61 |
| PolypPVT [9] | 81.21 | 73.78 | 94.37 | 59.34 | 88.05 | 79.4 |
| TransUNet [4] | 80.54 | 78.53 | 94.33 | 58.47 | 87.06 | 75.00 |
| SwinUNet [2] | 82.32 | 79.22 | 93.73 | 53.81 | 88.04 | 75.79 |
| MT-UNet [37] | 81.47 | 77.29 | 93.06 | 59.46 | 87.75 | 76.81 |
| MISSFormer [16] | 85.21 | 82.00 | 94.41 | 65.67 | 91.92 | 80.81 |
| PVT-CASCADE [28] | 82.23 | 80.37 | 94.08 | 64.43 | 90.1 | 83.69 |
| TransCASCADE [28] | 87.66 | 84.56 | 94.43 | 65.33 | 90.79 | 83.52 |
| Cascaded MERIT [29] | 87.71 | 84.62 | 95.02 | 70.74 | 91.98 | 85.17 |
| PVT-GCASCADE | 87.07 | 83.77 | 95.31 | 66.72 | 90.84 | 83.58 |
| (This study) | | | | | | |
| MERIT-GCASCADE | 88.01 | 84.83 | 95.38 | 69.73 | 91.92 | 83.63 |
| (This study) | | | | | | |

As shown in Table 1, the MERIT-GCASCADE system outperformed all the state-of-the-art CNN—and transformer-based 2D medical image segmentation methods, thus achieving the best average DICE score of 84.54%. The PVT-GCASCADE and MERIT-GCASCADE systems outperformed their counterparts PVT-CASCADE and Cascaded MERIT by 2.22% and 0.22% DICE scores, respectively with lower computational costs. Similarly, the PVT-GCASCADE and MERIT-GCASCADE systems outperformed their counterparts by 4.4 and 3.89 in HD95 distance. The MERIT-GCASCADE system had the lowest HD95 distance (10.38), which is 3.89 lower than the best SOA method Cascaded MERIT (HD95 of 14.27). The lower HD95 scores indicated that the G-CASCADE decoder can better locate the boundary of organs.

The G-CASCADE decoder also showed a boost in the DICE scores of individual organ segmentation. As shown in Table 1, the MERIT-GCASCADE system outperformed SOA methods on 5 out of 8 organs. The G-CASCADE decoder demonstrated better performance due to using graph convolution together with the transformer encoder.

Quantitative results on automated cardiac diagnosis challenge (ACDC) dataset. The study conducted another set of experiments on the MRI images of the ACDC dataset using the G-CASCADE-based systems (e.g., PVT-GCASCADE, MERIT-GCASCADE). Table 2 presents the average DICE scores of the PVT-GCASCADE and MERIT-GCASCADE systems along with other SOA methods. The study reported DICE scores for individual organs. The study got the results of SwinUNet from [28]. The study averaged G-CASCADE results over 5 runs. The best results are shown in bold.

TABLE 2

| | Average | | | |
|---|---|---|---|---|
| Architectures/Methods | Dice | RV | Myo | LV |
| R50 + UNet [4] | 87.55 | 87.10 | 80.63 | 94.92 |
| R50 + AttnUNet [4] | 86.75 | 87.58 | 79.20 | 93.47 |
| ViT + CUP [4] | 81.45 | 81.46 | 70.71 | 92.18 |
| R50 + ViT + CUP [4] | 87.57 | 86.07 | 81.88 | 94.75 |
| TransUNet [4] | 89.71 | 86.67 | 87.27 | 95.18 |
| SwinUNet [2] | 88.07 | 85.77 | 84.42 | 94.03 |
| MT-UNet [37] | 90.43 | 86.64 | 89.04 | 95.62 |
| MISSFormer [16] | 90.86 | 89.55 | 88.04 | 94.99 |
| PVT-CASCADE [28] | 91.46 | 89.97 | 88.9 | 95.50 |
| TransCASCADE [28] | 91.63 | 90.25 | 89.14 | 95.50 |
| Cascaded MERIT [29] | 91.85 | 90.23 | 89.53 | 95.80 |
| PVT-GCASCADE (This study) | 91.95 | 90.31 | 89.63 | 95.91 |
| MERIT-GCASCADE (This study) | 92.23 | 90.64 | 89.96 | 96.08 |

As shown in Table 2, the MERIT-GCASCADE system achieved the highest average DICE score of 92.23%, thus improving about 0.38% over Cascaded MERIT, though the G-CASCADE decoder had lower computational cost (shown in Table 5). The PVT-GCASCADE system gained 91.95% DICE score, which was also better than all other methods. Besides, both the PVT-GCASCADE and MERIT-GCASCADE systems had better DICE scores in all 3 organ segmentations.

Quantitative results on ISIC2018 dataset. Table 3 presents the average DICE scores of the PVT-GCASCADE and MERIT-GCASCADE systems, along with other SOA methods on the ISIC2018 dataset.

TABLE 3

Average

| Methods | DICE | mIoU |
|---|---|---|
| UNet [30] | 85.5 | 78.5 |
| UNet++ [49] | 80.9 | 72.9 |
| PraNet [11] | 87.5 | 78.7 |
| CaraNet [25] | 87.0 | 78.2 |
| TransUNet [4] | 88.0 | 80.9 |
| TransFuse [48] | 90.1 | 84.0 |
| UCTransNet [36] | 90.5 | 83.0 |
| PolypPVT [9] | 91.3 | 85.2 |
| PVT-CASCADE [28] | 91.1 | 84.9 |
| PVT-GCASCADE (This study) | 91.51 ± 0.61 | 86.53 ± 0.54 |

As shown in Table 3, the PVT-GCASCADE system achieved the best average DICE (91.51%) and mloU (86.53%) scores. The PVT-GCASCADE system outperformed its counterpart PVT-CASCADE by 0.4% DICE and 0.6% mloU scores.

Quantitative results on Polyp datasets. The study evaluated the performance and generalizability of the G-CASCADE-based system (e.g., PVT-GCASCADE) on 4 different polyp segmentation test sets (e.g., CVC-CLinicDB, Kvasir, ColonDB, EndoScene) among which 2 are unseen datasets collected from different labs. Table 4 displays the DICE and mloU scores of SoA methods along with the G-CASCADE decoder. The study took the results of UNet, UNet++, and PraNet from [11]. The study got the results of PolypPVT, SSFormerPVT, and UACANet from [28]. The study averaged PVT-GCASCADE results over 5 runs. The best results are shown in bold.

TABLE 4

| | CVC-ClinicDB | | Kvasir | | ColonDB | | EndoScene | |
|---|---|---|---|---|---|---|---|---|
| Methods | DICE | mIoU | DICE | mIoU | DICE | mIoU | DICE | mIoU |
| UNet [30] | 82.3 | 75.5 | 81.8 | 74.6 | 51.2 | 44.4 | 71.0 | 62.7 |
| UNet++ [49] | 79.4 | 72.9 | 82.1 | 74.3 | 48.3 | 41.0 | 70.7 | 62.4 |
| PraNet [11] | 89.9 | 84.9 | 89.8 | 84.0 | 71.2 | 64.0 | 87.1 | 79.7 |
| CaraNet [25] | 93.6 | 88.7 | 91.8 | 86.5 | 77.3 | 68.9 | 90.3 | 83.8 |
| UACANet-L [19] | 91.07 | 86.7 | 90.83 | 85.95 | 72.57 | 65.41 | 88.21 | 80.84 |
| SSFormerPVT [38] | 92.88 | 88.27 | 91.11 | 86.01 | 79.34 | 70.63 | 89.46 | 82.68 |
| PolypPVT [9] | 93.08 | 88.28 | 91.23 | 86.3 | 80.75 | 71.85 | 88.71 | 81.89 |
| PVT-CASCADE [28] | 94.34 | 89.98 | 92.58 | 87.76 | 82.54 | 74.53 | 90.47 | 83.79 |
| PVT-GCASCADE | 94.68 | 90.18 | 92.74 | 87.90 | 82.61 | 74.60 | 90.56 | 83.87 |
| (This study) | | | | | | | | |

As shown in Table 4, the G-CASCADE-based system significantly outperformed all other methods on both DICE and mloU scores. The G-CASCADE-based system outperformed the best CNN-based model UA-CANet by a large margin on unseen datasets (i.e., 9.8% DICE score improvement in ColonDB). Therefore, due to using transformers as a backbone network and the graph-based convolutional attention decoder, the PVT-GCASCADE system inherited the merits of transformers, GCNs, CNNs, and local attention, which makes them generalizable for unseen datasets.

Quantitative results on Retinal vessels segmentation datasets. The study conducted experiments on 2 retinal vessel segmentation datasets, such as DRIVE and CHASE_DB1. Table 5 shows the accuracy (i.e., ACC), sensitivity (i.e., Sen), specificity (i.e., Sp), DICE, and mloU scores (%) of SoA methods and the G-CASCADE-based systems (e.g., PVT-GCASCADE, MERIT-GCASCADE) operating on the DRIVE dataset. The study took the results of UNet, UNet++, Attention UNet, and FR-UNet from [22]. The study averaged all other results over 5 runs in the experimental setups. The best results are in bold.

TABLE 5

| Methods | Acc | Sen | Sp | DICE | IoU |
|---|---|---|---|---|---|
| UNet [30] | 96.78 | 80.57 | 98.33 | 81.41 | 68.64 |
| UNet++ [49] | 96.79 | 78.91 | 98.50 | 81.14 | 68.27 |
| Attention UNet [27] | 96.62 | 79.06 | 98.31 | 80.39 | 67.21 |
| FR-UNet [22] | 97.05 | 83.56 | 98.37 | 83.16 | 71.20 |
| PVTV2-b2 (only) [40] | 96.24 | 82.02 | 97.61 | 79.14 | 65.48 |
| PVT-CASCADE [28] | 96.79 | 83.07 | 98.10 | 81.73 | 69.10 |
| MERIT-CASCADE [29] | 96.89 | 82.94 | 98.22 | 82.21 | 69.08 |
| PVT-GCASCADE (this study) | 96.89 | 83.00 | 98.22 | 82.21 | 69.08 |
| MERIT-GCASCADE (this study) | 97.07 | 82.81 | 98.44 | 82.90 | 70.81 |

As shown in Table 5, the PVT-GCASCADE system showed a 0.37% improvement in DICE score over PVT-CASCADE in DRIVE dataset. Similarly, the MERIT-GCASCADE system exhibited a 0.69% improvement in DICE score in the DRIVE dataset.

Table 6 shows the accuracy (i.e., ACC), sensitivity (i.e., Sen), specificity (i.e., Sp), DICE, and mIoU scores (%) of SoA methods and the G-CASCADE-based systems (e.g., PVT-GCASCADE, MERIT-GCASCADE) operating on the CHASDE-DB1 dataset. The study took the results of UNet, UNet++, Attention UNet, and FR-UNet from [22]. The study averaged all other results over 5 runs in the experimental setups. The best results are in bold.

TABLE 6

| Methods | Acc | Sen | Sp | DICE | IoU |
|---|---|---|---|---|---|
| UNet [30] | 97.43 | 76.50 | 98.84 | 78.98 | 65.26 |
| UNet++ [49] | 97.39 | 83.57 | 98.32 | 80.15 | 66.88 |
| Attention UNet [27] | 97.30 | 83.84 | 98.20 | 79.64 | 66.17 |
| FR-UNet [22] | 97.48 | 87.98 | 98.14 | 81.51 | 68.82 |
| PVTV2-b2 (only) [40] | 97.25 | 85.07 | 98.07 | 79.58 | 66.12 |
| PVT-CASCADE [28] | 97.55 | 85.83 | 98.34 | 81.50 | 68.80 |
| MERIT-CASCADE [29] | 97.60 | 84.97 | 98.45 | 81.68 | 69.06 |
| PVT-GCASCADE (this study) | 97.71 | 85.84 | 98.51 | 82.51 | 70.24 |
| MERIT-GCASCADE (this study) | 97.76 | 84.93 | 98.62 | 82.67 | 70.50 |

As shown in Table 6, the PVT-GCASCADE system showed 1.01% improvement in DICE score over PVT-CASCADE CHASE_DB1 dataset. Similarly, the MERIT-GCASCADE system exhibited 0.99% improvement in DICE score in CHASE DB1 dataset.

From Tables 5 and 6, the study can conclude that the G-CASCADE-based systems showed competitive performance compared to the SoA approach, or even outperformed the SoA methods with significantly lower computational costs.

Although FR-UNet achieved a 0.26% better DICE score in the DRIVE dataset, it had a 1.16% lower DICE score in CHASE DB1 than the MERIT-GCASCADE system. Besides, FR-UNet split the retinal images into 48×48 pixels patches in a stride of 6 pixels during training, but the study used the whole retinal images during both training and inference. Consequently, the study had a significantly lower number of samples for training compared to FR-UNet. The study can conclude from the results that the G-CASCADE-based system equally performed well in retinal vessel segmentation.

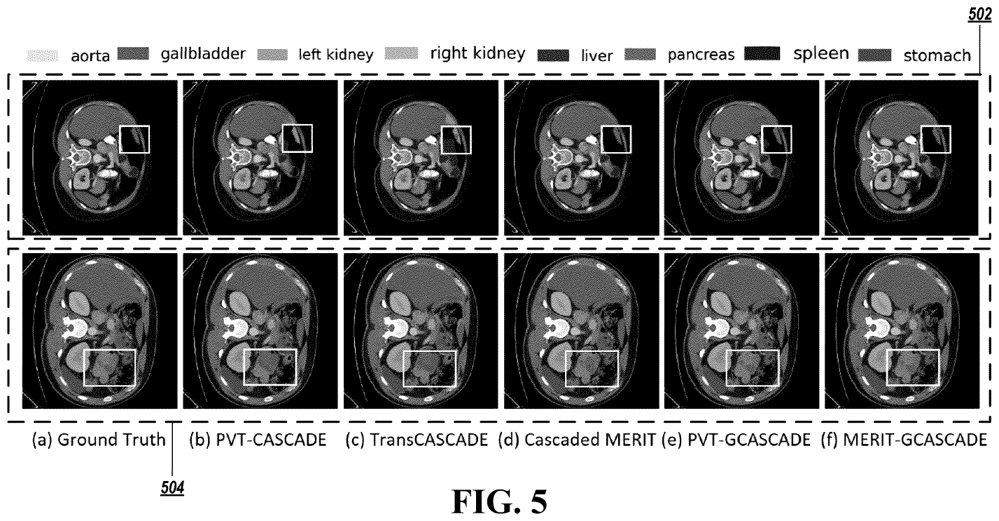

Qualitative results on Synapse multi-organ dataset. FIG. 5 shows the segmentation outputs of the G-CASCADE-based systems (e.g., PVT-GCASCADE, MERIT-GCASCADE) and 3 other state-of-the-art methods (e.g., PVT-CASCADE, TransCASCADE, Cascaded MERIT) on 2 sample images (e.g., 502 and 504). As shown in FIG. 5, the rectangular-boxed regions in both samples demonstrate that the MERIT-GCASCADE system segmented the organs with minimal false negative and false positive results. The PVT-GCASCADE and Cascaded MERIT systems showed comparable results. The PVT-GCASCADE system had false positives in the first sample 502 and had better segmentation in the second sample 504, whereas Cascaded MERIT provided better segmentation in the first sample 502, but it had larger false positives in the second sample 504. The Tran-sCASCADE and PVT-CASCADE systems provided larger incorrect segmentation outputs in both samples 502 and 504.

The study also performed a set of ablation experiments to examine the effect of different components or arrangements of components on the G-CASCADE-based system.

Effect of different components of G-CASCADE. The study carried out ablation experiments on the Synapse multi-organ dataset to evaluate the effectiveness of different components of the G-CASCADE-based system. The study used the same PVTv2-b2 backbone pre-trained on ImageNet and the same experimental settings for the Synapse multi-organ dataset in all experiments. The study removed different modules, such as Cascaded structure, graph convolution block (GCB), and spatial attention (SPA) from the G-CASCADE decoder and compared the results.

Table 7 shows the quantitative results of different components of G-CASCADE with PVTv2-b2 encoder on the Synapse multi-organ dataset. The study used additive aggregation for adding skip connections and an input resolution of 224×224 to get these results. The study averaged all results over 5 runs. The best results are shown in bold.

TABLE 7

| | | | | Number of | |
|---|---|---|---|---|---|
| Components | | | FLOPs | parameters | Average |
| Cascaded | GCB | SPA | (G) | (M) | DICE |
| No | No | No | 0 | 0 | 80.1 ± 0.2 |
| Yes | No | No | 0.102 | 0.225 | 81.1 ± 0.2 |
| Yes | No | Yes | 0.102 | 0.225 | 82.1 ± 0.3 |
| Yes | Yes | No | 0.341 | 1.78 | 83.0 ± 0.2 |
| Yes | Yes | Yes | 0.342 | 1.78 | 83.3 ± 0.2 |

As shown in Table 7, the cascaded structure of the G-CASCADE decoder improved performance over the non-cascaded decoder. GCB and SPA modules also helped improve performance. However, the use of both SPA and GCB modules together produced the best DICE score of 83.3%. As shown in Table 7, the DICE score was improved about 3.2% with 0.342G and 1.78M additional floating point operations (FLOPs) and parameters, respectively.

Effect of arrangement of GCB and SPA GCAM. The study conducted an ablation experiment to see the effect of the order of GCB and SPA in GCAM. Table 8 presents the experimental results of 2 different arrangements in GCAM on the Synapse multi-organ dataset. The study used PVTv2-b2 as the encoder to produce these results. The study averaged all the results over 5 runs. The best results are in bold.

TABLE 8

| Arrangements | DICE (%) |
|---|---|
| SPA → GCB | 82.93 ± 0.2 |
| GCB → SPA (this study) | 83.28 ± 0.2 |

As shown in Table 8, GCB followed by the SPA block performed better than SPA followed by GCB. Therefore, in the G-CASCADE decoder, the study used a GCB followed by an SPA block in each GCAM.

Comparison with the baseline decoder. Table 9 shows the experimental results with the computational complexity of the baseline CASCADE decoder and the G-CASCADE decoder. The study also reported the results of the original up convolution (UpConv) used in the CASCADE decoder and the modified efficient UCB. The study produced these results using a PVTv2-b2 encoder. The study averaged all the results over 5 runs. The best results are in bold.

TABLE 9

| | | FLOPs | Number of | DICE |
|---|---|---|---|---|
| Decoders | UCB | (G) | parameters | (%) |
| CASCADE | Original | 1.93 | 9.27 | 82.78 |
| CASCADE | Modified | 1.22 | 7.58 | 82.79 |
| G-CASCADE (this study) | Original | 1.06 | 3.47 | 83.15 |
| G-CASCADE (this study) | Modified | 0.342 | 1.78 | 83.28 |

As shown in Table 9, the modified UCB performed equally or better with lower FLOPs and parameters. The G-CASCADE decoder provided 0.5% better DICE score than the CASCADE decoder with 80.8% fewer parameters and 82.3% fewer FLOPs.

Effect of different skip-aggregations in G-CASCADE decoder. The study conducted some experiments to see the effect of Additive or Concatenation in aggregating upsampled features with skip-connections. Table 10 presents the results of the PVT-GCASCADE and MERIT-GCASCADE systems operating on the Synapse multi-organ dataset with Additive and concatenation aggregations. The study only reported the FLOPs and the number of parameters of the respective decoder. PVTV2-b2 encoder had 3.91G FLOPS and 24.86M parameters. Small MERIT encoder had 24.62G FLOPs and 129.38M parameters. The study averaged all the results over 5 runs. The best results are bold.

TABLE 10

| | | FLOPs | Number of | DICE |
|---|---|---|---|---|
| Architectures | Aggregation | (G) | parameters | (%) |
| PVT-GCASCADE | Addition | 0.342 | 1.78 | 83.28 |
| PVT-GCASCADE | Concatenation | 0.975 | 3.32 | 83.40 |
| MERIT-GCASCADE | Addition | 1.523 | 3.55 | 84.54 |
| MERIT-GCASCADE | Concatenation | 4.27 | 5.99 | 84.63 |

As shown in Table 10, Concatenation-based aggregation achieved marginally better DICE scores than Additive aggregation while having higher FLOPs and parameters. The reason behind this increase in computational complexity was the use of GCAM with the concatenated channels (i.e., 2× of original channels). Considering the lower computational complexity of Additive aggregation, the study used Additive aggregation in all of experiments.

Comparison among different graph convolutions in GCAM. Table 11 shows the experimental results of different graph convolutions in the GCAM block on the Synapse multi-organ dataset. The study used the PVTV2-b2 encoder and only reported the FLOPs and number of parameters of the decoder. The study averaged all the results over 5 runs. The best results are shown in bold.

TABLE 11

| | | Number of | |
|---|---|---|---|
| Graph convolutions | FLOPs (G) | parameters (M) | DICE (%) |
| GIN [46] | 0.313 | 1.59 | 82.22 |
| EdgeConv [41] | 0.957 | 1.78 | 82.81 |
| GraphSAGE [12] | 0.520 | 1.88 | 83.10 |
| Max-Relative [21] (this study) | 0.342 | 1.78 | 83.28 |

As shown in Table 11, Max-Relative (MR) graph convolution provided the best DICE score (83.28%) with only 0.342G FLOPs and 1.78M parameters. Although GIN had slightly lower FLOPs and parameters, it provided the lowest DICE score (82.22%). EdgeConv and GraphSAGE graph convolutions had lower DICE scores than the MR graph convolution with higher computational costs.

Overall computation complexity. Table 12 shows the comparison of overall computational complexity. The study used the PVTV2-b2 backbone with an input resolution of 224×224 in both the PVT-CASCADE system and PVT-GCASCADE system. The study used 2 Small Max ViT backbones with input resolutions of 256×256 and 224×224 in MERIT architectures. The study averaged all the results over 5 runs. The best results are in bold.

TABLE 12

| | | Number of | |
|---|---|---|---|
| Architectures | FLOPs (G) | parameters (M) | DICE (%) |
| PVT-CASCADE | 5.84 | 34.13 | 83.28 |
| PVT-GCASCADE | 4.252 | 26.64 | 83.40 |
| MERIT-CASCADE | 33.31 | 147.86 | 84.54 |
| MERIT-GCASCADE | 26.143 | 132.93 | 84.63 |

As shown in Table 12, overall computational complexity depended on the number of parameters and FLOPs of the encoder backbones. The study implemented the decoder on top of PVTV2-b2 and Small Max ViT backbones. The PVT-GCASCADE system had 4.252G FLOPs and 26.64M parameters, which was 1.588G and 7.49M lower than the corresponding PVT-CASCADE architecture. Due to the larger size of 2 Small Max ViT backbones in MERIT-CASCADE architecture (i.e., 33.31G FLOPs and 147.86M parameters), the MERIT-GCASCADE system (i.e., 26.143G FLOPs and 132.93M parameters) was also larger in size. In both cases, the savings in FLOPs and parameters came only from the G-CASCADE decoder. The G-CASCADE decoder can be plugged into other hierarchical encoders; if a lightweight encoder was used, the total computational cost can be reduced.

Influence of input resolution. Table 13 presents the quantitative segmentation performance of the PVT-GCASCADE network with different input resolutions on the Synapse multi-organ dataset. The study conducted experiments with 3 input resolutions such as 224×224, 256×256, and 384×384. The study averaged all the results over 5 runs.

TABLE 13

| Input resolutions | DICE (%) | mIoU (%) | HD95 (%) |
|---|---|---|---|
| 224 × 224 | 83.28 | 73.91 | 15.83 |
| 256 × 256 | 84.21 | 75.32 | 14.58 |
| 384 × 384 | 86.01 | 78.10 | 13.67 |

As shown in Table 13, performance improved in all 3 evaluation metrics for higher input resolutions. The study got the best DICE and mIoU 86.01% and 78.10%, respectively, with the input resolution of 384×384.

### Embodiment #2—Efficient Multi-Scale Convolutional Attention Decoding Image Segmentation System (i.e., EMCAD-Based System, EMCAD Decoder)

The study implemented EMCAD-based systems (e.g., PVT-EMCAD-B0, PVT-EMCAD-B2) and compared them against the state-of-the-art (SoA) methods when operating on different datasets. EMCAD employs a multi-scale depth-wise convolution block, which is key for capturing diverse scale information within feature maps, a critical factor for precision in medical image segmentation. The use of depth-wise convolutions instead of standard 3×3 convolution blocks makes EMCAD notably efficient.

Experiments reveal that EMCAD surpasses the recent CASCADE decoder in DICE scores with 79.4% fewer parameters and 80.3% fewer FLOPs. The extensive experiments also confirm EMCAD's superior performance compared to SOTA methods across 12 public datasets covering six different 2D medical image segmentation tasks. EMCAD's compatibility with smaller encoders makes it an excellent fit for point-of-care applications while maintaining high performance.

Datasets. To evaluate the performance of the EMCAD decoder, the study carried out experiments across 12 datasets that belong to 6 medical image segmentation tasks (e.g., polyp, abdomen organ, cardiac organ, skin lesion, breast cancer, cell nuclei/structure).

For polyp segmentation, the study used 5 polyp segmentation datasets: Kvasir [29′] (1,000 images), ClinicDB [3′] (612 images), ColonDB [51′] (379 images), ETIS [51′] (196 images), and BKAI [40′] (1,000 images). These datasets contained images from different imaging centers/clinics, having greater diversity in image nature as well as the size and shape of polyps.

For abdomen segmentation, the study used the Synapse multi-organ dataset. This dataset contained 30 abdominal CT scans, which had 3,779 axial contrast-enhanced slices. Each CT scan had 85-198 slices of 512×512 pixels. Following TransUNet [8′], the study used the same 18 scans for training (2,212 axial slices) and 12 scans for validation. The study segmented only 8 abdominal organs, namely the aorta, gallbladder (GB), left kidney (KL), right kidney (KR), liver, pancreas (PC), spleen (SP), and stomach (SM).

For cardiac organ segmentation, the study used an automated cardiac diagnosis challenge (ACDC) dataset. It contained 100 cardiac MRI scans having three sub-organs, namely right ventricle (RV), myocardium (Myo), and left ventricle (LV). Following TransUNet [8′], the study used 70 cases (1,930 axial slices) for training, 10 for validation, and 20 for testing.

For skin lesion segmentation, the study used the ISIC17 dataset [15′] (2,000 training, 150 validation, and 600 testing images) and the ISIC18 dataset [14′] (2,594 images).

For breast cancer segmentation, the study used the breast cancer ultrasound image dataset (BUSI) [1′] dataset for breast cancer segmentation. Following [50′], the study used 647 (437 benign and 210 malignant) images from this dataset.

For cell nuclei/structure segmentation, the study used the data science bowl 2018 (DSB18) [4′] (670 images) and electromagnetic (EM) [6′] (30 images) datasets of biological imaging for cell nuclei/structure segmentation.

The study used a train-val-test split of 80:10:10 in ClinicDB, Kvasir, ColonDB, ETIS, BKAI, ISIC18, DSB18, EM, and BUSI datasets. For ISIC17, the study used the official train-val-test sets provided by a third party.

Evaluation metrics. The study used the 95% Hausdorff Distance (HD95), DICE score, and mloU score, defined in Equations 20-22 to evaluate the performance of the EMCAD-based and SoA systems on all the datasets.

Implementation details. The study implemented the EMCAD-based system and conducted experiments using Pytorch 1.11.0 on a single NVIDIA RTX A6000 GPU with 48 GB of memory. The study utilized ImageNet [16′], pretrained PVTv2-b0, and pretrained PVTv2-b2 [56′] as encoders. In the MSDC of the EMCAD decoder, the study set the multi-scale kernels [1′], [3′], [5′] through an ablation study. The study used the parallel arrangement of depth-wise convolutions in all experiments. The models were trained using the AdamW optimizer [36′] with a learning rate and weight decay of le ⁻⁴.

The study trained for 200 epochs with a batch size of 16, except for Synapse multi-organ (300 epochs, batch size 6) and ACDC cardiac organ (400 epochs, batch size 12), saving the best model based on the DICE score. The study resized images to 352×352 and used a multi-scale {0.75, 1.0, 1.25} training strategy with a gradient clip limit of 0.5 for ClinicDB [3′], Kvasir [29′], ColonDB [51′], ETIS [51′], BKAI [40′], ISIC17 [15′], and ISIC18 [15′], while the study resized images to 256×256 for BUSI [1′], EM [6′], and DSB 18 [4′]. For Synapse and ACDC datasets, images were resized to 224×224, with random rotation and flipping augmentations, optimizing a combined Cross-entropy (0.3) and DICE (0.7) loss. For binary segmentation, the study utilized the combined weighted BinaryCrossEntropy (BCE) and weighted IoU loss function.

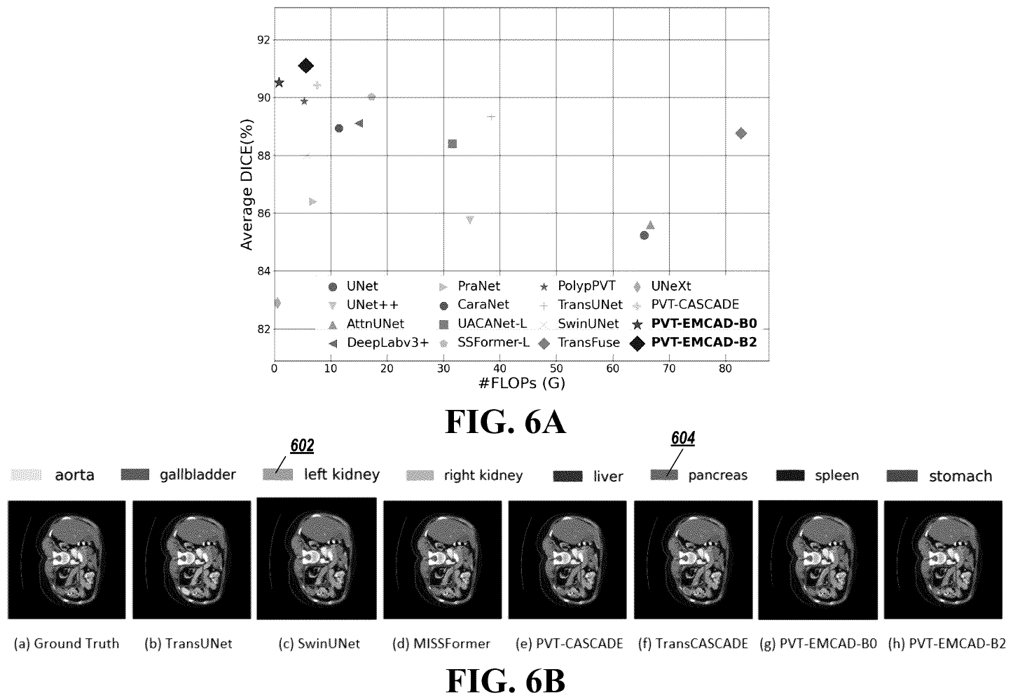

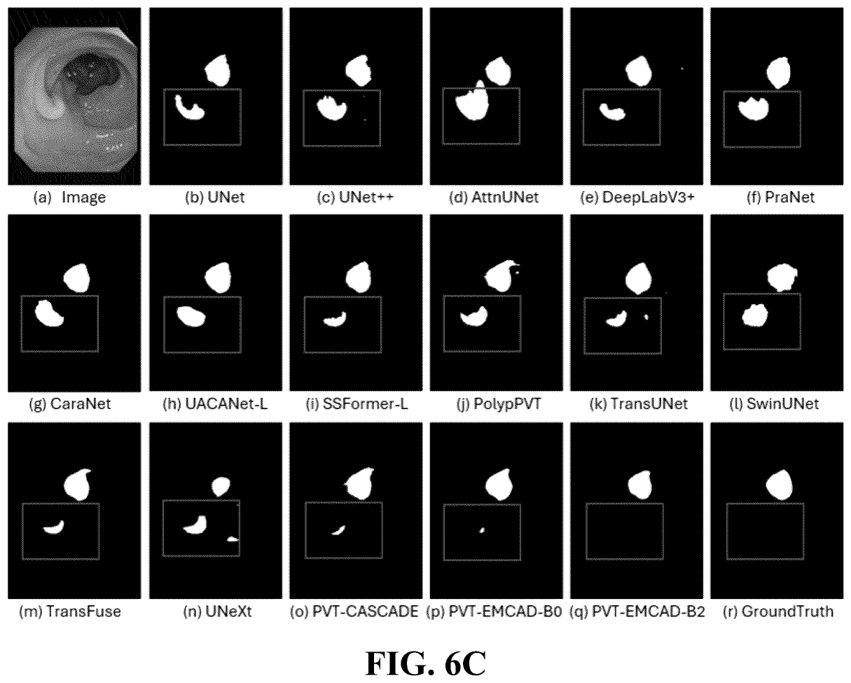

Quantitative results on binary medical image segmentation datasets. FIG. 6A and Table 14 shows the quantitative results for the EMCAD-based systems (e.g., PVT-EMCAD-B0, PVT-EMCAD-B2), and different SoA methods on 10 binary medical image segmentation datasets. The study reproduced the results of SoA methods using their publicly available implementation with the train-val-test splits of 80:10:10. The study reported the number of floating point operations of all the methods for 256×256 inputs, except Swin-UNet (224×224). The study averaged all the results over 5 runs. Best results are shown in bold.

TABLE 14

| | Number of | FLOPs | Polyp | | | | |
|---|---|---|---|---|---|---|---|
| Methods | parameters | (G) | Clinic | Colon | ETIS | Kvasir | BKAI |
| UNet [44′] | 34.53M | 65.53G | 92.11 | 83.95 | 76.85 | 82.87 | 85.05 |
| UNet++ [62′] | 9.16M | 34.65G | 92.17 | 87.88 | 77.40 | 83.36 | 84.07 |
| AttnUNet [41′] | 34.88M | 66.64G | 92.20 | 86.46 | 76.84 | 83.49 | 84.07 |
| DeepLabv3+ [10′] | 39.76M | 14.92G | 93.24 | 91.92 | 90.73 | 89.06 | 89.74 |
| PraNet [20′] | 32.55M | 6.93G | 91.71 | 89.16 | 83.84 | 84.82 | 85.56 |
| CaraNet [38′] | 46.64M | 11.48G | 94.08 | 91.19 | 90.25 | 89.74 | 89.71 |
| UACANet-L [30′] | 69.16M | 31.51G | 94.16 | 91.02 | 89.77 | 90.17 | 90.35 |
| SSFormer-L [54′] | 66.22M | 17.28G | 94.18 | 92.11 | 90.16 | 91.47 | 91.14 |
| PolypPVT [17′] | 25.11M | 5.30G | 94.13 | 91.53 | 89.93 | 91.56 | 91.17 |
| TransUNet [8′] | 105.32M | 38.52G | 93.90 | 91.63 | 87.79 | 91.08 | 89.17 |
| SwinUNet [5′] | 27.17M | 6.2G | 92.42 | 89.27 | 85.10 | 89.59 | 87.61 |
| TransFuse [61′] | 143.74M | 82.71G | 93.62 | 90.35 | 86.91 | 90.24 | 87.47 |
| UNext [50′] | 1.47M | 0.57G | 90.20 | 83.84 | 74.03 | 77.88 | 77.93 |
| PVT-CASCADE [42′] | 34.12M | 7.62G | 94.53 | 91.60 | 91.03 | 92.05 | 92.14 |
| PVT-EMCAD-B0 | 3.92M | 0.84G | 94.60 | 91.71 | 91.65 | 91.95 | 91.30 |
| (this study) | | | | | | | |
| PVT-EMCAD-B2 | 26.76M | 5.6G | 95.21 | 92.31 | 92.29 | 92.75 | 92.96 |
| (this study) | | | | | | | |

| | Skin Lesion | | Cell | | | |
|---|---|---|---|---|---|---|
| Methods | ISIC17 | ISIC18 | DSB18 | EM | BUSI | Avg |
| UNet [44′] | 83.07 | 86.67 | 92.23 | 95.46 | 74.04 | 85.23 |
| UNet++ [62′] | 82.98 | 87.46 | 91.97 | 95.48 | 74.76 | 85.75 |
| AttnUNet [41′] | 83.66 | 87.05 | 92.22 | 95.55 | 74.48 | 85.60 |
| DeepLabv3+ [10′] | 83.84 | 88.64 | 92.14 | 94.96 | 76.81 | 89.11 |
| PraNet [20′] | 83.03 | 88.56 | 89.89 | 92.37 | 75.14 | 86.41 |
| CaraNet [38′] | 85.02 | 90.18 | 89.15 | 92.78 | 77.34 | 88.94 |
| UACANet-L [30′] | 83.72 | 89.76 | 88.86 | 89.28 | 76.96 | 88.41 |
| SSFormer-L [54′] | 85.28 | 90.25 | 92.03 | 94.95 | 78.76 | 90.03 |
| PolypPVT [17′] | 85.56 | 90.36 | 90.69 | 94.40 | 79.35 | 89.87 |
| TransUNet [8′] | 85.00 | 89.16 | 92.04 | 95.27 | 78.30 | 89.33 |
| SwinUNet [5′] | 83.97 | 89.26 | 91.03 | 94.47 | 77.38 | 88.01 |
| TransFuse [61′] | 84.89 | 89.62 | 90.85 | 94.35 | 79.36 | 88.77 |
| UNext [50′] | 82.74 | 87.78 | 86.01 | 93.81 | 74.71 | 82.89 |
| PVT-CASCADE [42′] | 85.50 | 90.41 | 92.35 | 95.42 | 79.21 | 90.42 |
| PVT-EMCAD-B0 | 85.67 | 90.70 | 92.46 | 95.35 | 79.80 | 90.52 |
| (this study) | | | | | | |
| PVT-EMCAD-B2 | 85.95 | 90.96 | 92.74 | 95.53 | 80.25 | 91.10 |
| (this study) | | | | | | |

As shown in Table 14, the PVT-EMCAD-B2 system attained the highest average DICE score (91.10%) with only 26.76M parameters and 5.6G FLOPs. The multi-scale depth-wise convolution in the EMCAD decoder, combined with the transformer encoder, contributed to these performance gains.

In 5 polyp segmentation datasets, the PVT-EMCAD-B2 system surpassed all SoA methods as shown in Table 14. The PVT-EMCAD-B2 system achieved DICE score improvements of 1.08%, 0.78%, 2.36%, 1.19%, and 1.79% over PolypPVT in ClinicDB, ColonDB, ETIS, Kvasir, and BKAI-IGI, despite having more parameters and FLOPs. The smallest model UNeXt exhibited the worst performance in all 5 polyp segmentation datasets. The study's smaller model with only 3.92M parameters and 0.84G FLOPS also outperformed all the methods except PVT-CASCADE (in Kvasir and BKAI-IGH) and SSFormer-L (in ColonDB), which achieved the best performance among SoA methods. In conclusion, the PVT-EMCAD-B2 system achieved the SoA results in these 5 polyp segmentation datasets.

In skin lesion segmentation datasets (e.g., ISIC17 and ISIC18), as shown in Table 14, the PVT-EMCAD-B2 system achieved DICE scores of 85.95% and 90.96%, surpassing DeepLabV3+ by 2.11% and 2.32%. The PVT-EMCAD-B2 system also beat the nearest method PVT-CASCADE by 0.45% and 0.55% in ISIC17 and ISIC18, respectively, though the PVT-EMCAD-B2 system was more efficient than CASCADE. The PVT-EMCAD-B0 system also showed huge potential in point care applications like skin lesion segmentation with only 3.92M parameters and 0.84G FLOPS.

The study used DSB18 [4′] for cell nuclei and EM [6′] for cell structure segmentation datasets to evaluate the the EMCAD-based system's effectiveness in biological imaging. As shown in Table 14, the PVT-EMCAD-B2 system set a SoA benchmark in cell nuclei segmentation on DSB18, outperforming DeepLabv3+, TransFuse, and PVT-CASCADE. On the EM dataset, the PVT-EMCAD-B2 system secured the second-best DICE score (95.53%), offering lower computational costs than the top-performing AttnUNet (95.55%).

The study also conducted experiments on the BUSI dataset for breast cancer segmentation in ultrasound images. As shown in Table 14, the PVT-EMCAD-B2 system achieved the DICE score (80.25%) on this dataset. Furthermore, the PVT-EMCAD-B0 system outperformed the computationally similar method UNeXt by a notable margin of 5.54%.

Quantitative results on abdomen organ segmentation datasets. Table 15 shows the quantitative results of abdomen organ segmentation on the Synapse multi-organ dataset for the EMCAD-based systems (e.g., PVT-EMCAD-B0, PVT-EMCAD-B2) and SoA methods. The study reported the DICE scores for individual organs. The study took results of UNet, AttnUNet, PolypPVT, SSFormerPVT, TransUNet, and SwinUNet from [42′]. ↑(↓) denotes the higher (lower), the better. ‘−’ means missing data from the source. The study averaged the EMCAD results over 5 runs. The best results are shown in bold.

TABLE 15

| | Average | | | | |
|---|---|---|---|---|---|
| Architectures | DICE↑ | HD95↓ | mIoU↑ | Aorta | GB |
| UNet [44′] | 70.11 | 44.69 | 59.39 | 84.00 | 56.70 |
| AttnUNet [41′] | 71.70 | 34.47 | 61.38 | 82.61 | 61.94 |
| R50 + UNet [8′] | 74.68 | 36.87 | — | 84.18 | 62.84 |
| R50 + AttnUNet [8′] | 75.57 | 36.97 | — | 55.92 | 63.91 |
| SSFormer [54′] | 78.01 | 25.72 | 67.23 | 82.78 | 63.74 |
| PolypPVT [17′] | 78.08 | 25.61 | 67.43 | 82.34 | 66.14 |
| TransUNet [8′] | 77.61 | 26.9 | 67.32 | 86.56 | 60.43 |
| SwinUNet [5′] | 77.58 | 27.32 | 66.88 | 81.76 | 65.95 |
| MT-UNet [53′] | 78.59 | 26.59 | — | 87.92 | 64.99 |
| MISSFormer [25′] | 81.96 | 18.20 | — | 86.99 | 68.65 |
| PVT-CASCADE [42′] | 81.06 | 20.23 | 70.88 | 83.01 | 70.59 |
| TransCASCADE [42′] | 82.68 | 17.34 | 73.48 | 86.63 | 68.48 |
| PVT-EMCAD-B0 | 81.97 | 17.39 | 72.64 | 87.21 | 66.62 |
| (this study) | | | | | |
| PVT-EMCAD-B2 | 83.63 | 15.68 | 74.65 | 88.14 | 68.87 |
| (this study) | | | | | |

| Architectures | KL | KR | Liver | PC | SP | SM |
|---|---|---|---|---|---|---|
| UNet [44′] | 72.41 | 62.64 | 86.98 | 48.73 | 81.48 | 67.96 |
| AttnUNet [41′] | 76.07 | 70.42 | 87.54 | 46.70 | 80.67 | 67.66 |
| R50 + UNet [8′] | 79.19 | 71.29 | 93.35 | 48.23 | 84.41 | 73.92 |
| R50 + AttnUNet [8′] | 79.20 | 72.71 | 93.56 | 49.37 | 87.19 | 74.95 |
| SSFormer [54′] | 80.72 | 78.11 | 93.53 | 61.53 | 87.07 | 76.61 |
| PolypPVT [17′] | 81.21 | 73.78 | 94.37 | 59.34 | 88.05 | 79.4 |
| TransUNet [8′] | 80.54 | 78.53 | 94.33 | 58.47 | 87.06 | 75.00 |
| SwinUNet [5′] | 82.32 | 79.22 | 93.73 | 53.81 | 88.04 | 75.79 |
| MT-UNet [53′] | 81.47 | 77.29 | 93.06 | 59.46 | 87.75 | 76.81 |
| MISSFormer [25′] | 85.21 | 82.00 | 94.41 | 65.67 | 91.92 | 80.81 |
| PVT-CASCADE [42′] | 82.23 | 80.37 | 94.08 | 64.43 | 90.1 | 83.69 |
| TransCASCADE [42′] | 87.66 | 84.56 | 94.43 | 65.33 | 90.79 | 83.52 |
| PVT-EMCAD-B0 | 87.48 | 83.96 | 94.57 | 62.00 | 92.66 | 81.22 |
| (this study) | | | | | | |
| PVT-EMCAD-B2 | 88.08 | 84.10 | 95.26 | 68.51 | 92.17 | 83.92 |
| (this study) | | | | | | |

As shown in Table 15, the PVT-EMCAD-B2 system excelled in abdomen organ segmentation on the Synapse multi-organ dataset, achieving the highest average DICE score of 83.63% and surpassing all SoA CNN—and transformer-based methods. The PVT-EMCAD-B2 outperformed PVT-CASCADE by 2.57% in the DICE score and 4.55 in HD95 distance, indicating superior organ boundary location. Overall, the EMCAD-based systems boosted individual organ segmentation, significantly outperforming SoA methods on 6 of 8 organs.

Quantitative results on cardiac organ segmentation datasets. Table 16 shows the DICE scores of the PVT-EMCAD-B2 and PVT-EMCAD-B0 systems, along with other SoA methods, on the MRI images of the ACDC dataset for cardiac organ segmentation.

TABLE 16

| Methods | Average DICE | RV | Myo | LV |
|---|---|---|---|---|
| R50 + UNet [8′] | 87.55 | 87.10 | 80.63 | 94.92 |
| R50 + AttnUNet [8′] | 86.75 | 87.58 | 79.20 | 93.47 |
| ViT + CUP [8′] | 81.45 | 81.46 | 70.71 | 92.18 |
| R50 + VIT + CUP [8′] | 87.57 | 86.07 | 81.88 | 94.75 |
| TransUNet [8′] | 89.71 | 86.67 | 87.27 | 95.18 |
| SwinUNet [5′] | 88.07 | 85.77 | 84.42 | 94.03 |
| MT-UNet [53′] | 90.43 | 86.64 | 89.04 | 95.62 |
| MISSFormer [25′] | 90.86 | 89.55 | 88.04 | 94.99 |
| PVT-CASCADE [42′] | 91.46 | 89.97 | 88.9 | 95.50 |
| TransCASCADE [42′] | 91.63 | 90.25 | 89.14 | 95.50 |
| Cascaded MERIT [43′] | 91.85 | 90.23 | 89.53 | 95.80 |
| PVT-EMCAD-B0 | 91.34 ± 0.2 | 89.37 | 88.99 | 95.65 |
| (this study) | | | | |
| PVT-EMCAD-B2 | 92.12 ± 0.2 | 90.65 | 89.68 | 96.02 |
| (this study) | | | | |

As shown in Table 16, the PVT-EMCAD-B2 system achieved the highest average DICE score of 92.12%, thus improving about 0.27% over Cascaded MERIT, though the EMCAD-based network had significantly lower computational cost. Besides, the PVT-EMCAD-B2 system had better DICE scores in all 3 organ segmentations.

Qualitative results on Synapse multi-organ datasets. FIG. 6B shows the qualitative results of multi-organ segmentation on the Synapse multi-organ dataset for the EMCAD-based systems (e.g., PVT-EMCAD-B0, PVT-EMCAD-B2) and SoA methods.

As shown in FIG. 6B, most of the methods faced challenges segmenting the left kidney 602 and part of the pancreas 604. However, the PVT-EMCAD-B0 system (shown in subpanel g) and the PVT-EMCADB2 system (shown in subpanel h) can segment those organs more accurately (shown in rectangular-boxed regions) with lower computational costs.

FIG. 6C shows the qualitative results of polyp segmentation on the ClinicDB dataset for the EMCAD-based systems (e.g., PVT-EMCAD-B0, PVT-EMCAD-B2) and SoA methods. As shown in FIG. 6C, predicted segmentation outputs of the PVT-EMCAD-B0 system (shown in subpanel p) and the PVT-EMCAD-B2 system (shown in subpanel q) had strong overlaps with the GroundTruth mask (shown in subpanel r), while existing SoA methods exhibited false segmentation of polyp (shown in rectangular-boxed regions).

The study also performed a set of ablation experiments to examine the effect of different components or arrangements of components on the EMCAD-based system.

Effect of different components of EMCAD-based system. The study conducted a set of experiments on the Synapse multi-organ dataset to understand the effect of different components of the EMCAD decoder. The study started with only the encoder and added different modules such as Cascaded structure, large-kernel grouped attention gates (LGAG), and multiscale convolutional attention modules (MSCAM) to understand their effect.

Table 17 shows the effect of different components of the EMCAD decoder on the Synapse multi-organ dataset. The study reported the number of floating point operations (FLOPs) for input resolutions of 224×224 and 256×256. The study averaged all results over 5 runs. The best results are shown in bold.

TABLE 17

| | | | | Number of | |
|---|---|---|---|---|---|
| Components | | | FLOPs | parameters | Average |
| Cascaded | LGAG | MSCAM | (G) | (M) | DICE |
| No | No | No | 0 | 0 | 80.1 ± 0.2 |
| Yes | No | No | 0.100 | 0.224 | 81.08 ± 0.2 |
| Yes | Yes | No | 0.108 | 0.235 | 81.92 ± 0.2 |
| Yes | No | Yes | 0.373 | 1.898 | 82.86 ± 0.3 |
| Yes | Yes | Yes | 0.381 | 1.91 | 83.63 ± 0.3 |

As shown in Table 17, the cascaded structure of the decoder helped to improve performance over the non-cascaded one. The incorporation of LGAG and MSCAM improved performance; however, MSCAM proved to be more effective. When both the LGAG and MSCAM modules were used together, it produced the best DICE score of 83.63%. There was about 3.53% improvement in the DICE score with an additional 0.381G FLOPs and 1.91M parameters.

Effect of multi-scale kernels in MSCAM. The study conducted another set of experiments on Synapse multi-organ and ClinicDB datasets to understand the effect of different multi-scale kernels used for depth-wise convolutions in multi-scale depth-wise convolution (MSDC).

Table 18 shows the effect of multi-scale kernels in the depth-wise convolution of MSDC on ClinicDB and Synapse multi-organ datasets. The study averaged all the results over 5 runs. The best results are highlighted in bold.

TABLE 18

| Convolution Kernel | [1] | [3] | [5] | [1, 3] | [3, 3] | [1, 3, 5] | [3, 3, 3] |
|---|---|---|---|---|---|---|---|
| Synapse | 82.43 | 82.79 | 82.74 | 82.98 | 82.81 | 83.63 | 82.92 |
| ClinicDB | 94.81 | 94.90 | 94.98 | 95.13 | 95.06 | 95.21 | 95.15 |

| Convolution Kernel | [3, 5, 7] | [1, 3, 5, 7] | [1, 3, 5, 7, 9] |
|---|---|---|---|
| Synapse | 83.11 | 83.57 | 83.34 |
| ClinicDB | 95.03 | 95.18 | 95.07 |

As shown in Table 18, performance improved from 1×1 to 3×3 kernel. When 1×1 kernel was used together with 3×3, the performance improved more than when using them alone. However, when two 3×3 kernels were used together, performance dropped. The incorporation of a 5×5 kernel with 1×1 and 3×3 kernels further improved the performance, and it achieved the best results in both Synapse multi-organ and ClinicDB datasets. When the study added additional larger kernels (e.g., 7×7, 9×9), the performance of both datasets dropped. Based on these empirical observations, the study chose [1, 3, 5] kernels in all the experiments.

Comparison with the baseline decoder. Table 19 shows the performance of the EMCAD decoders and baseline decoders (e.g., CASCADE) on the Synapse multi-organ dataset. The study only reported the number of floating point operations (FLOPs) (with input resolution of 224×224) and the number of parameters of the decoders. The study averaged all the results over 5 runs. The best results are shown in bold.

TABLE 19

| | | | Number of | |
|---|---|---|---|---|
| | | FLOPs | parameters | DICE |
| Encoders | Decoders | (G) | (M) | (%) |
| PVTv2-B0 | CASCADE | 0.439 | 2.32 | 80.54 |
| PVTv2-B0 | EMCAD (this study) | 0.102 | 0.507 | 81.97 |
| PVTv2-B2 | CASCADE | 1.93 | 9.27 | 82.78 |
| PVTv2-B2 | EMCAD (this study) | 0.381 | 1.91 | 83.63 |

As shown in Table 19, the EMCAD decoder with PVTv2-b2 required 80.3% fewer FLOPs and 79.4% fewer parameters to outperform (by 0.85%) the respective CASCADE decoder. Similarly, the EMCAD decoder with PVTv2-B0 achieved a 1.43% better DICE score than the CASCADE decoder with 78.1% fewer parameters and 74.9% fewer FLOPs.

Parallel and sequential depth-wise convolution. The study conducted another set of experiments to decide whether to use multi-scale depth-wise convolutions in parallel or sequential.

Table 20 shows the results of parallel and sequential depth-wise convolution in multi-scale depth-wise convolution (MSDC) on Synapse multi-organ and ClinicDB datasets for EMCAD-based systems (e.g., PVT-EMCAD-B0, PVT-EMCAD-B2). The study averaged all the results over 5 runs. The best results are in bold.

TABLE 20

| | Depth-wise | | |
|---|---|---|---|
| Architectures | convolution | Synapse | ClinicDB |
| PVT-EMCAD-B0 | Sequential | 81.82 ± 0.3 | 94.57 ± 0.2 |
| PVT-EMCAD-B0 | Parallel | 81.97 ± 0.2 | 94.60 ± 0.2 |
| PVT-EMCAD-B2 | Sequential | 83.54 ± 0.3 | 95.15 ± 0.3 |
| PVT-EMCAD-B2 | Parallel | 83.63 ± 0.2 | 95.21 ± 0.2 |

As shown in Table 20, there was no significant impact of the arrangements, though the parallel convolutions provided a slightly improved performance (0.03% to 0.15%). The study also observed higher standard deviations among runs in the case of sequential convolutions. Hence, in all the experiments, the study used multi-scale depth-wise convolutions in parallel.

Effectiveness of the large-kernel grouped attention gate (LGAG) over attention gate (AG). Table 21 shows the experimental results of the EMCAD-based systems (e.g., PVT-EMCAD-B0, PVT-EMCAD-B2) with AG [41′] and LGAG on Synapse multi-organ dataset. The study reported the total number of parameters and floating point operations (FLOPs) of 3 AG/LGAGs in the EMCAD decoder for an input resolution of 256×256. The study averaged all results over 5 runs. The best results are in bold.

TABLE 21

| Architectures | Module | Parameters (K) | FLOPs (M) | Synapse |
|---|---|---|---|---|
| PVT-EMCAD-B0 | AG | 31.62 | 15.91 | 81.74 |
| PVT-EMCAD-B0 | LGAG | 5.51 | 5.24 | 81.97 |
| PVT-EMCAD-B2 | AG | 124.68 | 61.68 | 83.51 |
| PVT-EMCAD-B2 | LGAG | 11.01 | 10.47 | 83.63 |

As shown in Table 21, the LGAG achieved better DICE scores with lower number of parameters (82.57% for PVT-EMCAD-B0 and 91.17% for PVT-EMCAD-B2) and FLOPs (67.06% for PVT-EMCAD-B0 and 83.03% for PVT-EMCAD-B2) than AG. The reduction in the number of parameters and FLOPs was bigger for the larger models. Therefore, the LGAG demonstrated improved scalability with models that had a greater number of channels, yielding enhanced DICE scores.

Effect of transfer learning from ImageNet pretrained weights. The study conducted experiments on the Synapse multi-organ dataset to show the effect of transfer learning from the ImageNet pretrained encoder. Table 22 shows the experimental results of transfer learning from ImageNet pretained weights on EMCAD-based systems (e.g., PVT-EMCAD-B0, PVT-EMCAD-B2) operating on Synapse multi-organ dataset. ↑(↓) denotes that the higher (lower) the better. The study averaged all results over 5 runs. The best results are in bold.

TABLE 22

| | | Average | | |
|---|---|---|---|---|
| Architectures | Pretrain | DICE↑ | HD95↓ | mIoU↑ |
| PVT-EMCAD-B0 | No | 77.47 | 19.93 | 66.72 |
| PVT-EMCAD-B0 | Yes | 81.97 | 17.39 | 72.64 |
| PVT-EMCAD-B2 | No | 80.18 | 18.83 | 70.21 |
| PVT-EMCAD-B2 | Yes | 83.63 | 15.68 | 74.65 |

| Architectures | Pretrain | Aorta | GB | KL | KR | Liver | PC | SP | SM |
|---|---|---|---|---|---|---|---|---|---|
| PVT-EMCAD-B0 | No | 81.96 | 69.41 | 83.88 | 74.82 | 93.45 | 54.41 | 88.97 | 72.85 |
| PVT-EMCAD-B0 | Yes | 87.21 | 66.62 | 87.48 | 83.96 | 94.57 | 62.00 | 92.66 | 81.22 |
| PVT-EMCAD-B2 | No | 85.98 | 68.10 | 84.62 | 79.93 | 93.96 | 61.61 | 90.99 | 76.23 |
| PVT-EMCAD-B2 | Yes | 88.14 | 68.87 | 88.08 | 84.10 | 95.26 | 68.51 | 92.17 | 83.92 |

As shown in Table 22, transfer learning from ImageNet pre-trained PVT-v2 encoders boosted the performance. Specifically, for the PVT-EMCAD-B0 system, the DICE, mloU, and HD95 scores were improved by 4.5%, 5.92%, and 2.54, respectively. Likewise, for the PVT-EMCAD-B2 system, the DICE, mloU, and HD95 scores were improved by 3.45%, 4.44%, and 3.15, respectively. Transfer learning had a comparatively greater impact on the smaller PVT-EMCAD-B0 model than the larger PVT-EMCAD-B2 model. For individual organs, transfer learning boosted the performance of all organ segmentation except the Gallbladder (GB).

Effect of deep supervision. The study conducted an ablation experiment that dropped the Deep Supervision (DS). Table 23 shows the effect of deep supervision (DS) on the performance of the EMCAD-based systems (e.g., PVT-EMCAD-B0, PVT-EMCAD-B2) when operating on EM, BUSI, Clinic, Kvasir, ISIC18, Synapse, and ACDC datasets.

TABLE 23

| DS | EM | BUSI | Clinic | Kvasir | ISIC18 | Synapse | ACDC |
|---|---|---|---|---|---|---|---|
| No | 95.74 | 79.64 | 94.96 | 92.51 | 90.74 | 82.03 | 92.08 |
| Yes | 95.53 | 80.25 | 95.21 | 92.75 | 90.96 | 83.63 | 92.12 |

As shown in Table 23, the PVT-EMCAD-B2 system with DS achieved better DICE scores in 6 out of 7 datasets. Among all the datasets, the DS had the largest impact on the Synapse multi-organ dataset.

Effect of input resolutions. Table 24 shows the experimental results of the EMCAD-based systems (e.g., PVT-EMCAD-B0, PVT-EMCAD-B2) with different input resolutions on the Synapse multi-organ dataset. The study averaged all results over 5 runs.

TABLE 24

| Architectures | Resolutions | FLOPs (G) | DICE |
|---|---|---|---|
| PVT-EMCAD-B0 | 224 × 224 | 0.64 | 81.97 |
| PVT-EMCAD-B0 | 256 × 256 | 0.84 | 82.63 |
| PVT-EMCAD-B0 | 384 × 384 | 1.89 | 84.81 |
| PVT-EMCAD-B0 | 512 × 512 | 3.36 | 85.52 |
| PVT-EMCAD-B2 | 224 × 224 | 4.29 | 83.63 |
| PVT-EMCAD-B2 | 256 × 256 | 5.60 | 84.47 |
| PVT-EMCAD-B2 | 384 × 384 | 12.59 | 85.78 |
| PVT-EMCAD-B2 | 512 × 512 | 22.39 | 86.53 |

As shown in Table 24, the DICE scores improved with the increase in input resolution. However, these improvements in the DICE score came with an increment in the number of FLOPs. The PVT-EMCAD-B0 system achieved an 85.52% DICE score with only 3.36G FLOPs when using 512×512 inputs. On the other hand, the PVT-EMCAD-B2 system achieved the best DICE score (86.53%) with 22.39G FLOPs when using 512×512 inputs. The PVT-EMCAD-B2 system with 5.60G FLOPs, when using 256×256 inputs, showed a 1.05% lower DICE score than the PVT-EMCAD-B0 system with 3.36G FLOPS. Therefore, the PVT-EMCAD-B0 system was more suitable for larger input resolutions than the PVT-EMCAD-B2 system.

### DISCUSSION

Discussion #1. Automatic medical image segmentation plays a crucial role in the diagnosis, treatment planning, and post-treatment evaluation of various diseases; this involves classifying pixels and generating segmentation maps to identify lesions, tumors, or organs. Convolutional neural networks (CNNs) have been extensively utilized for medical image segmentation tasks [30], [27], [49], [15], [11], [26]. Among them, the U-shaped networks such as UNct [30], UNet++ [49], UNet 3+ [15], and DC-UNet exhibit reasonable performance and produce high-resolution segmentation maps. Additionally, researchers have incorporated attention modules into their architectures [27], [6], to enhance feature maps and improve pixel-level classification of medical images by capturing salient features. Although these attention-based methods have shown improved performance, they still struggle to capture long-range dependencies [28].

Recently, vision transformers has shown great promise in capturing long-range dependencies among pixels and demonstrated improved performance, particularly for medical image segmentation [4], [2], [9], [38], [28], [29], [48], [36]. The self-attention (SA) mechanism used in transformers learns correlations between input patches; this enables capturing the long-range dependencies among pixels. Recently, hierarchical vision transformers such as the Swin transformer [23], the pyramid vision transformer (PVT) [39], Max ViT [34], MERIT [29], have been introduced to enhance performance. These hierarchical vision transformers are effective in medical image segmentation tasks [4], [2], [9], [38], [28], [29]. As self-attention modules employed in transformers have limited capacity to learn (local) spatial relationships among pixels [7], [17], some methods [44], [42], [40], [9], [38], [28], incorporate local convolutional attention modules in the decoder. However, due to the locality of convolution operations, these methods have difficulties in capturing long-range correlations among pixels.

To overcome the aforementioned limitations, the study introduced a Graph-based Cascaded Convolutional Attention Decoder (i.e., G-CASCADE-based system, G-CASCADE decoder) using graph convolutions. More precisely, G-CASCADE enhances the feature maps by preserving long-range attention due to the global receptive field of the graph convolution operation while incorporating local attention through the spatial attention mechanism.

Discussion #2. In the realm of medical diagnostics and therapeutic strategies, automated segmentation of medical images is vital, as it classifies pixels to identify critical regions such as lesions, tumors, or entire organs. A variety of U-shaped convolutional neural network (CNN) architectures [20′], [24′], [37′], [41′], [44′], [62′], notably UNet [44′], UNet++ [62′], UNet3+ [24′], and nnU-Net [19′], have become standard techniques for this purpose, achieving high-quality, high-resolution segmentation output. Attention mechanisms [12′], [17′], [20′], [41′], [57′] have also been integrated into these models to enhance feature maps and improve pixel-level classification. Although attention-based models have shown improved performance, they still face challenges due to the computationally expensive convolutional blocks that are used in conjunction with attention mechanisms.

Recently, vision transformers [18′] have shown promise in medical image segmentation tasks [5′], [8′], [17′], [42′], [43′], [52′], [54′], [61′] by capturing long-range dependencies among pixels through Self-attention (SA) mechanisms. Hierarchical vision transformers like Swin [34′], PVT [55′], [56′], Max ViT [49′], MERIT [43′], ConvFormer [33′], and MetaFormer [59′] have been introduced to further improve the performance in this field.

While the SA excels at capturing global information, it is less adept at understanding the local spatial context [13′], [28′]. To address this limitation, some approaches have integrated local convolutional attention within the decoders to better grasp spatial details. Nevertheless, these methods can still be computationally demanding because they frequently employ costly convolutional blocks. This limits their applicability to real-world scenarios where computational resources are restricted.

To address the aforementioned limitations, the study introduce efficient multi-layer convolutional attention decoding (EMCAD) image segmentation system using a multi-scale depth-wise convolution block. More precisely, EMCAD enhances the feature maps via efficient multi-scale convolutions, while incorporating complex spatial relationships and local attention through the use of channel, spatial, and grouped (large-kernel) gated attention mechanisms.

### CONCLUSION

The construction and arrangement of the systems and methods as shown in the various implementations are illustrative only. Although only a few implementations have been described in detail in this disclosure, many modifications are possible (e.g., variations in sizes, dimensions, structures, shapes, and proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative implementations. Other substitutions, modifications, changes, and omissions may be made in the design, operating conditions, and arrangement of the implementations without departing from the scope of the present disclosure.

The present disclosure contemplates methods, systems, and program products on any machine-readable media for accomplishing various operations. The implementation of the present disclosure may be implemented using existing computer processors, or by a special purpose computer processor for an appropriate system, incorporated for this or another purpose, or by a hardwired system. Implementations within the scope of the present disclosure include program products including machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures, and which can be accessed by a general purpose or special purpose computer or other machine with a processor.

When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general-purpose computer, special purpose computer, or special purpose processing machines to perform a certain function or group of functions.

Although the figures show a specific order of method steps, the order of the steps may differ from what is depicted. Also, two or more steps may be performed concurrently or with partial concurrence. Such variation will depend on the software and hardware systems chosen and on designer choice. All such variations are within the scope of the disclosure. Likewise, software implementations could be accomplished with programming techniques with rule-based logic and other logic to accomplish the various connection steps, processing steps, comparison steps and decision steps.

Each and every feature described herein, and each and every combination of two or more of such features, is included within the scope of the present invention, provided that the features included in such a combination are not mutually inconsistent.

Although example embodiments of the disclosed technology are explained in detail herein, it is to be understood that other embodiments are contemplated. Accordingly, it is not intended that the disclosed technology be limited in its scope to the details of construction and arrangement of components set forth in the following description or illustrated in the drawings. The disclosed technology is capable of other embodiments and of being practiced or carried out in various ways.

It must also be noted that, as used in the specification and the appended claims, the singular forms “a,” “an” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

While the methods and systems have been described in connection with certain embodiments and specific examples, it is not intended that the scope be limited to the particular embodiments set forth, as the embodiments herein are intended in all respects to be illustrative rather than restrictive.

The following patents, applications, and publications, as listed below and throughout this document, are hereby incorporated by reference in their entirety herein.

### REFERENCE LIST #1

[1] Jorge Bernal, F Javier Sanchez, Gloria Fernandez-Esparrach, Debora Gil, Cristina Rodriguez, and Fernando Vilarino. Wm-dova maps for accurate polyp highlighting in colonoscopy: Validation vs. saliency maps from physicians. Computerized Medical Imaging and Graphics, 43:99-111, 2015.
[2] Hu Cao, Yueyue Wang, Joy Chen, Dongsheng Jiang, Xi-aopeng Zhang, Qi Tian, and Manning Wang. Swin-unet: Unet-like pure transformer for medical image segmentation. arXiv preprint arXiv: 2105.05537, 2021.
[3] Adrian Carballal, Francisco J Novoa, Carlos Fernandez-Lozano, Marcos Garcia-Guimaraes, Guillermo Aldama-Lopez, Ramon Calvino-Santos, Jose Manuel Vazquez-Rodriguez, and Alejandro Pazos. Automatic multiscale vas-cular image segmentation algorithm for coronary angiogra-phy. Biomedical Signal Processing and Control, 46:1-9, 2018.
[4] Jieneng Chen, Yongyi Lu, Qihang Yu, Xiangde Luo, Ehsan Adeli, Yan Wang, Le Lu, Alan L Yuille, and Yuyin Zhou. Transunet: Transformers make strong encoders for medical image segmentation. arXiv preprint arXiv: 2102.04306, 2021.
[5] Long Chen, Hanwang Zhang, Jun Xiao, Liqiang Nie, Jian Shao, Wei Liu, and Tat-Seng Chua. Sca-cnn: Spatial and channel-wise attention in convolutional networks for image captioning. In Proceedings of the IEEE Conference on Com-puter Vision and Pattern Recognition (CVPR), pages 5659-5667, 2017.
[6] Shuhan Chen, Xiuli Tan, Ben Wang, and Xuelong Hu. Re-verse attention for salient object detection. In Proceedings of the European Conference on Computer Vision (ECCV), pages 234-250, 2018.
[7] Xiangxiang Chu, Zhi Tian, Bo Zhang, Xinlong Wang, Xi-aolin Wei, Huaxia Xia, and Chunhua Shen. Conditional po-sitional encodings for vision transformers. arXiv preprint arXiv: 2102.10882, 2021.
[8] Noel Codella, Veronica Rotemberg, Philipp Tschandl, M Emre Celebi, Stephen Dusza, David Gutman, Brian Helba, Aadi Kalloo, Konstantinos Liopyris, Michael Marchetti, et al. Skin lesion analysis toward melanoma detection 2018: A challenge hosted by the interna-tional skin imaging collaboration (isic). arXiv preprint arXiv: 1902.03368, 2019.
[9] Bo Dong, Wenhai Wang, Deng-Ping Fan, Jinpeng Li, Huazhu Fu, and Ling Shao. Polyppvt: Polyp segmen-tation with pyramid vision transformers. arXiv preprint arXiv: 2108.06932, 2021.
[10] Alexey Dosovitskiy, Lucas Beyer, Alexander Kolesnikov, Dirk Weissenborn, Xiaohua Zhai, Thomas Unterthiner, Mostafa Dehghani, Matthias Minderer, Georg Heigold, Sylvain Gelly, et al. An image is worth 16×16 words: Trans-formers for image recognition at scale. arXiv preprint arXiv: 2010.11929, 2020.
[11] Deng-Ping Fan, Ge-Peng Ji, Tao Zhou, Geng Chen, Huazhu Fu, Jianbing Shen, and Ling Shao. Pranet: Parallel reverse attention network for polyp segmentation. In International Conference on Medical Image Computing and Computer-Assisted Intervention (MICCAI), pages 263-273. Springer, 2020.
[12] Will Hamilton, Zhitao Ying, and Jure Leskovec. Inductive representation learning on large graphs. Advances in Neural Information Processing Systems, 30, 2017.
[13] Kai Han, Yunhe Wang, Jianyuan Guo, Yehui Tang, and En-hua Wu. Vision gnn: An image is worth graph of nodes. arXiv preprint arXiv: 2206.00272, 2022.
[14] Jie Hu, Li Shen, and Gang Sun. Squeeze-and-excitation net-works. In Proceedings of the IEEE Conference on Computer Vision and Pattern Recognition (CVPR), pages 7132-7141, 2018.
[15] Huimin Huang, Lanfen Lin, Ruofeng Tong, Hongjie Hu, Qiaowei Zhang, Yutaro Iwamoto, Xianhua Han, Yen-Wei Chen, and Jian Wu. Unet 3+: A full-scale connected unet for medical image segmentation. In International Confer-ence on Acoustics, Speech and Signal Processing (ICASSP), pages 1055-1059. IEEE, 2020.
[16] Xiaohong Huang, Zhifang Deng, Dandan Li, and Xueguang Yuan. Missformer: An effective medical image segmentation transformer. arXiv preprint arXiv: 2109.07162, 2021.
[17] Md Amirul Islam, Sen Jia, and Neil D B Bruce. How much position information do convolutional neural networks en-code? arXiv preprint arXiv: 2001.08248, 2020.
[18] Debesh Jha, Pia H Smedsrud, Michael A Riegler, Poal Halvorsen, Thomas de Lange, Dag Johansen, and Hoavard D Johansen. Kvasir-seg: A segmented polyp dataset. In International Conference on Multimedia Modeling, pages 451-462. Springer, 2020.
[19] Taehun Kim, Hyemin Lee, and Daijin Kim. Uacanet: Uncertainty augmented context attention for polyp segmentation. In Proceedings of the 29th ACM International Conference on Multimedia, pages 2167-2175, 2021.
[20] Loic Landrieu and Martin Simonovsky. Large-scale point cloud semantic segmentation with superpoint graphs. In Pro-ceedings of the IEEE Conference on Computer Vision and Pattern Recognition (CVPR), pages 4558-4567, 2018.
[21] Guohao Li, Matthias Muller, Ali Thabet, and Bernard Ghanem. Deepgens: Can gens go as deep as cnns? In Proceedings of the IEEE/CVF International Conference on Computer Vision (ICCV), pages 9267-9276, 2019.
[22] Wentao Liu, Huihua Yang, Tong Tian, Zhiwei Cao, Xipeng Pan, Weijin Xu, Yang Jin, and Feng Gao. Full-resolution net-work and dual-threshold iteration for retinal vessel and coro-nary angiograph segmentation. IEEE Journal of Biomedical and Health Informatics, 26 (9): 4623-4634, 2022.
[23] Ze Liu, Yutong Lin, Yue Cao, Han Hu, Yixuan Wei, Zheng Zhang, Stephen Lin, and Baining Guo. Swin transformer: Hierarchical vision transformer using shifted windows. In Proceedings of the IEEE/CVF International Conference on Computer Vision (ICCV), pages 10012-10022, 2021.
[24] Ilya Loshchilov and Frank Hutter. Decoupled weight decay regularization. arXiv preprint arXiv: 1711.05101, 2017.
[25] Ange Lou, Shuyue Guan, Hanseok Ko, and Murray H Loew. Caranet: context axial reverse attention network for segmen-tation of small medical objects. In Medical Imaging 2022: Image Processing, volume 12032, pages 81-92. SPIE, 2022.
[26] Ange Lou, Shuyue Guan, and Murray Loew. Dc-unet: re-thinking the u-net architecture with dual channel efficient cnn for medical image segmentation. In Medical Imaging 2021: Image Processing, volume 11596, pages 758-768. SPIE, 2021.
[27] Ozan Oktay, Jo Schlemper, Loic Le Folgoc, Matthew Lee, Mattias Heinrich, Kazunari Misawa, Kensaku Mori, Steven McDonagh, Nils Y Hammerla, Bernhard Kainz, et al. Atten-tion u-net: Learning where to look for the pancreas. arXiv preprint arXiv: 1804.03999, 2018.
[28] Md Mostafijur Rahman and Radu Marculescu. Medical im-age segmentation via cascaded attention decoding. In Pro-ceedings of the IEEE/CVF Winter Conference on Applications of Computer Vision (WACV), pages 6222-6231 January-uary 2023.
[29] Md Mostafijur Rahman and Radu Marculescu. Multi-scale hierarchical vision transformer with cascaded attention de-coding for medical image segmentation. In Medical Imaging with Deep Learning, 2023.
[30] Olaf Ronneberger, Philipp Fischer, and Thomas Brox. U-net: Convolutional networks for biomedical image segmentation. In International Conference on Medical Image Computing and Computer-Assisted Intervention (MICCAI), pages 234-241. Springer, 2015.
[31] Joes Staal, Michael D Abramoff, Meindert Niemeijer, Max A Viergever, and Bram Van Ginneken. Ridge-based vessel seg-mentation in color images of the retina. IEEE transactions on medical imaging, 23 (4): 501-509, 2004.
[32] Nima Tajbakhsh, Suryakanth R Gurudu, and Jianming Liang. Automated polyp detection in colonoscopy videos using shape and context information. IEEE Transactions on Medical Imaging, 35 (2): 630-644, 2015.
[33] Feilong Tang, Qiming Huang, Jinfeng Wang, Xianxu Hou, Jionglong Su, and Jingxin Liu. Duat: Dual-aggregation transformer network for medical image segmentation. arXiv preprint arXiv: 2212.11677, 2022.
[34] Zhengzhong Tu, Hossein Talebi, Han Zhang, Feng Yang, Peyman Milanfar, Alan Bovik, and Yinxiao Li. Maxvit: Multi-axis vision transformer. In Proceedings of the European Conference on Computer Vision (ECCV), pages 459-479. Springer, 2022.
[35] David Vazquez, Jorge Bernal, F Javier S'anchez, Gloria Fernandez-Esparrach, Antonio M Lopez, Adriana Romero, Michal Drozdzal, and Aaron Courville. A benchmark for endoluminal scene segmentation of colonoscopy images. Jour-nal of Healthcare Engineering, 2017, 2017.
[36] Haonan Wang, Peng Cao, Jiaqi Wang, and Osmar R Zaiane. Uctransnet: rethinking the skip connections in u-net from a channel-wise perspective with transformer. In Proceedings of the AAAI Conference on Artificial Intelligence, volume 36, pages 2441-2449, 2022.
[37] Hongyi Wang, Shiao Xie, Lanfen Lin, Yutaro Iwamoto, Xian-Hua Han, Yen-Wei Chen, and Ruofeng Tong. Mixed transformer u-net for medical image segmentation. In International Conference on Acoustics, Speech and Signal Pro-cessing (ICASSP), pages 2390-2394. IEEE, 2022.
J[38] infeng Wang, Qiming Huang, Feilong Tang, Jia Meng, Jion-glong Su, and Sifan Song. Stepwise feature fusion: Local guides global. arXiv preprint arXiv: 2203.03635, 2022.
[39] Wenhai Wang, Enze Xie, Xiang Li, Deng-Ping Fan, Kaitao Song, Ding Liang, Tong Lu, Ping Luo, and Ling Shao. Pyramid vision transformer: A versatile backbone for dense prediction without convolutions. In Proceedings of the IEEE/CVF International Conference on Computer Vision (ICCV), pages 568-578, 2021.
[40] Wenhai Wang, Enze Xie, Xiang Li, Deng-Ping Fan, Kaitao Song, Ding Liang, Tong Lu, Ping Luo, and Ling Shao. Pvt v2: Improved baselines with pyramid vision transformer. Computational Visual Media, 8 (3): 415-424, 2022.
[41] Yue Wang, Yongbin Sun, Ziwei Liu, Sanjay E Sarma, Michael M Bronstein, and Justin M Solomon. Dynamic graph cnn for learning on point clouds. Acm Transactions On Graphics, 38 (5): 1-12, 2019.
[42] Zhendong Wang, Xiaodong Cun, Jianmin Bao, Wengang Zhou, Jianzhuang Liu, and Houqiang Li. Uformer: A general u-shaped transformer for image restoration. In Proceedings of the IEEE/CVF Conference on Computer Vision and Pattern Recognition (CVPR), pages 17683-17693, 2022.
[43] Sanghyun Woo, Jongchan Park, Joon-Young Lee, and In So Kweon. Cham: Convolutional block attention module. In Proceedings of the European Conference on Computer Vision (ECCV), pages 3-19, 2018.
[44] Enze Xie, Wenhai Wang, Zhiding Yu, Anima Anandkumar, Jose M Alvarez, and Ping Luo. Segformer: Simple and efficient design for semantic segmentation with transformers. Advances in Neural Information Processing Systems, 34:12077-12090, 2021.
[45] Danfei Xu, Yuke Zhu, Christopher B Choy, and Li Fei-Fei. Scene graph generation by iterative message passing. In Pro-ceedings of the IEEE Conference on Computer Vision and Pattern Recognition (CVPR), pages 5410-5419, 2017.
[46] Keyulu Xu, Weihua Hu, Jure Leskovec, and Stefanie Jegelka. How powerful are graph neural networks? arXiv preprint arXiv: 1810.00826, 2018.
[47] Sijie Yan, Yuanjun Xiong, and Dahua Lin. Spatial tempo-ral graph convolutional networks for skeleton-based action recognition. In Proceedings of the AAAI Conference on Artificial Intelligence, volume 32, 2018.
[48] Yundong Zhang, Huiye Liu, and Qiang Hu. Transfuse: Fus-ing transformers and cnns for medical image segmentation. In International Conference on Medical Image Computing and Computer-Assisted Intervention, pages 14-24. Springer, 2021.
[49] Zongwei Zhou, Md Mahfuzur Rahman Siddiquee, Nima Tajbakhsh, and Jianming Liang. Unet++: A nested u-net ar-chitecture for medical image segmentation. In Deep Learning in Medical Image Analysis and Multimodal Learning for Clinical Decision Support, pages 3-11. Springer, 2018.

### REFERENCE LIST #2

[1′] Walid Al-Dhabyani, Mohammed Gomaa, Hussien Khaled, and Aly Fahmy. Dataset of breast ultrasound images. Data in brief, 28:104863, 2020.
[2′] Vijay Badrinarayanan, Alex Kendall, and Roberto Cipolla. Segnet: A deep convolutional encoder-decoder architecture for image segmentation. IEEE Trans. Pattern Anal. Mach. Intell., 39 (12): 2481-2495, 2017.
[3′] Jorge Bernal, F Javier S'anchez, Gloria Fernandez-Esparrach, Debora Gil, Cristina Rodr'iguez, and Fernando Vilari no. Wm-dova maps for accurate polyp highlighting in colonoscopy: Validation vs. saliency maps from physicians. Comput. Med. Imaging Graph., 43:99-111, 2015.
[4′] Juan C Caicedo, Allen Goodman, Kyle W Karhohs, Beth A Cimini, Jeanelle Ackerman, Marzieh Haghighi, CherKeng Heng, Tim Becker, Minh Doan, Claire McQuin, et al. Nu-cleus segmentation across imaging experiments: the 2018 data science bowl. Nature methods, 16 (12): 1247-1253, 2019.
[5′] Hu Cao, Yueyue Wang, Joy Chen, Dongsheng Jiang, Xi-aopeng Zhang, Qi Tian, and Manning Wang. Swin-unet: Unet-like pure transformer for medical image segmentation. arXiv preprint arXiv: 2105.05537, 2021.
[6′] Albert Cardona, Stephan Saalfeld, Stephan Preibisch, Ben-jamin Schmid, Anchi Cheng, Jim Pulokas, Pavel Tomancak, and Volker Hartenstein. An integrated micro- and macroar-chitectural analysis of the drosophila brain by computer-assisted serial section electron microscopy. PLOS biology, 8 (10): e 1000502, 2010.
[7′] Gongping Chen, Lei Li, Yu Dai, Jianxun Zhang, and Moi Hoon Yap. Aau-net: an adaptive attention u-net for breast lesions segmentation in ultrasound images. IEEE Trans. Med. Imaging, 2022.
[8′] Jieneng Chen, Yongyi Lu, Qihang Yu, Xiangde Luo, Ehsan Adeli, Yan Wang, Le Lu, Alan L Yuille, and Yuyin Zhou. Transunet: Transformers make strong encoders for medical image segmentation. arXiv preprint arXiv: 2102.04306, 2021.
[9′] Long Chen, Hanwang Zhang, Jun Xiao, Liqiang Nie, Jian Shao, Wei Liu, and Tat-Seng Chua. Sca-cnn: Spatial and channel-wise attention in convolutional networks for image captioning. In IEEE Conf. Comput. Vis. Pattern Recog., pages 5659-5667, 2017.
[10′] Liang-Chieh Chen, George Papandreou, Iasonas Kokkinos, Kevin Murphy, and Alan L Yuille. Deeplab: Semantic image segmentation with deep convolutional nets, atrous convolu-tion, and fully connected crfs. IEEE Trans. Pattern Anal. Mach. Intell., 40 (4): 834-848, 2017.
[11′] Liang-Chieh Chen, Yukun Zhu, George Papandreou, Florian Schroff, and Hartwig Adam. Encoder-decoder with atrous separable convolution for semantic image segmentation. In Eur. Conf. Comput. Vis., pages 801-818, 2018.
[12′] Shuhan Chen, Xiuli Tan, Ben Wang, and Xuelong Hu. Re-verse attention for salient object detection. In Eur. Conf. Comput. Vis., pages 234-250, 2018.
[13′] Xiangxiang Chu, Zhi Tian, Bo Zhang, Xinlong Wang, Xi-aolin Wei, Huaxia Xia, and Chunhua Shen. Conditional positional encodings for vision transformers. arXiv preprint arXiv: 2102.10882, 2021.
[14′] Noel Codella, Veronica Rotemberg, Philipp Tschandl, M Emre Celebi, Stephen Dusza, David Gutman, Brian Helba, Aadi Kalloo, Konstantinos Liopyris, Michael Marchetti, et al. Skin lesion analysis toward melanoma detection 2018: A challenge hosted by the interna-tional skin imaging collaboration (isic). arXiv preprint arXiv: 1902.03368, 2019.
[15′] Noel C F Codella, David Gutman, M Emre Celebi, Brian Helba, Michael A Marchetti, Stephen W Dusza, Aadi Kalloo, Konstantinos Liopyris, Nabin Mishra, Harald Kit-tler, et al. Skin lesion analysis toward melanoma detection: A challenge at the 2017 international symposium on biomed-ical imaging (isbi), hosted by the international skin imaging collaboration (isic). In IEEE Int. Symp. Biomed. Imaging, pages 168-172. IEEE, 2018.
[16′] Jia Deng, Wei Dong, Richard Socher, Li-Jia Li, Kai Li, and Li Fei-Fei. Imagenet: A large-scale hierarchical image database. In IEEE Conf. Comput. Vis. Pattern Recog., pages 248-255. Ieee, 2009.
[17′] Bo Dong, Wenhai Wang, Deng-Ping Fan, Jinpeng Li, Huazhu Fu, and Ling Shao. Polyppvt: Polyp segmen-tation with pyramid vision transformers. arXiv preprint arXiv: 2108.06932, 2021.
[18′] Alexey Dosovitskiy, Lucas Beyer, Alexander Kolesnikov, Dirk Weissenborn, Xiaohua Zhai, Thomas Unterthiner, Mostafa Dehghani, Matthias Minderer, Georg Heigold, Sylvain Gelly, et al. An image is worth 16×16 words: Trans-formers for image recognition at scale. arXiv preprint arXiv: 2010.11929, 2020.
[19′] Isensee et al. nnu-net: a self-configuring method for deep learning-based biomedical image segmentation. Nature methods, 18 (2): 203-211, 2021.
[20′] Deng-Ping Fan, Ge-Peng Ji, Tao Zhou, Geng Chen, Huazhu Fu, Jianbing Shen, and Ling Shao. Pranet: Parallel re-verse attention network for polyp segmentation. In Int. Conf. Med. Image Comput. Comput. Assist. Interv., pages 263-273. Springer, 2020.
[21′] Kaiming He, Xiangyu Zhang, Shaoqing Ren, and Jian Sun. Deep residual learning for image recognition. In IEEE Conf. Comput. Vis. Pattern Recog., pages 770-778, 2016.
[22′] Andrew G Howard, Menglong Zhu, Bo Chen, Dmitry Kalenichenko, Weijun Wang, Tobias Weyand, Marco An-dreetto, and Hartwig Adam. Mobilenets: Efficient convolutional neural networks for mobile vision applications. arXiv preprint arXiv: 1704.04861, 2017.
[23′] Jie Hu, Li Shen, and Gang Sun. Squeeze-and-excitation net-works. In IEEE Conf. Comput. Vis. Pattern Recog., pages 7132-7141, 2018.
[24′] Huimin Huang, Lanfen Lin, Ruofeng Tong, Hongjie Hu, Qiaowei Zhang, Yutaro Iwamoto, Xianhua Han, Yen-Wei Chen, and Jian Wu. Unet 3+: A full-scale connected unet for medical image segmentation. In ICASSP, pages 1055-1059. IEEE, 2020.
[25′] Xiaohong Huang, Zhifang Deng, Dandan Li, and Xueguang Yuan. Missformer: An effective medical image segmentation transformer. arXiv preprint arXiv: 2109.07162, 2021.
[26′] Nabil Ibtehaz and Daisuke Kihara. Acc-unet: A com-pletely convolutional unet model for the 2020s. In Int. Conf. Med. Image Comput. Comput. Assist. Interv., pages 692-702. Springer, 2023.
[27′] Sergey Ioffe and Christian Szegedy. Batch normalization: Accelerating deep network training by reducing internal co-variate shift. In Int. Conf. Mach. Learn., pages 448-456. pmlr, 2015.
[28′] Md Amirul Islam, Sen Jia, and Neil D B Bruce. How much position information do convolutional neural networks en-code? arXiv preprint arXiv: 2001.08248, 2020. 1
[29′] Debesh Jha, Pia H Smedsrud, Michael A Riegler, Pal Halvorsen, Thomas de Lange, Dag Johansen, and Havard D Johansen. Kvasir-seg: A segmented polyp dataset. In Int. Conf. Multimedia Model., pages 451-462. Springer, 2020.
[30′] Taehun Kim, Hyemin Lee, and Daijin Kim. Uacanet: Uncertainty augmented context attention for polyp segmentation. In ACM Int. Conf. Multimedia, pages 2167-2175, 2021.
[31′] Alex Krizhevsky and Geoff Hinton. Convolutional deep be-lief networks on cifar-10. Unpublished manuscript, 40 (7): 1-9, 2010.
[32′] Alex Krizhevsky, Ilya Sutskever, and Geoffrey E Hinton. Imagenet classification with deep convolutional neural net-works. Adv. Neural Inform. Process. Syst., 25, 2012.
[33′] Xian Lin, Zengqiang Yan, Xianbo Deng, Chuansheng Zheng, and Li Yu. Convformer: Plug-and-play cnn-style transformers for improving medical image segmentation. In Int. Conf. Med. Image Comput. Comput. Assist. Interv., pages 642-651. Springer, 2023.
[34′] Ze Liu, Yutong Lin, Yue Cao, Han Hu, Yixuan Wei, Zheng Zhang, Stephen Lin, and Baining Guo. Swin transformer: Hierarchical vision transformer using shifted windows. In Int. Conf. Comput. Vis., pages 10012-10022, 2021.
[35′] Zhuang Liu, Hanzi Mao, Chao-Yuan Wu, Christoph Feicht-enhofer, Trevor Darrell, and Saining Xie. A convnet for the 2020s. In IEEE Conf. Comput. Vis. Pattern Recog., pages 11976-11986, 2022.
[36′] Ilya Loshchilov and Frank Hutter. Decoupled weight decay regularization. arXiv preprint arXiv: 1711.05101, 2017.
[37′] Ange Lou, Shuyue Guan, and Murray Loew. Dc-unet: re-thinking the u-net architecture with dual channel efficient cnn for medical image segmentation. In Med. Imaging 2021: Image Process., pages 758-768. SPIE, 2021.
[38′] Ange Lou, Shuyue Guan, Hanseok Ko, and Murray H Loew. Caranet: context axial reverse attention network for segmen-tation of small medical objects. In Med. Imaging 2022: Im-age Process., pages 81-92. SPIE, 2022.
[39′] Vinod Nair and Geoffrey E Hinton. Rectified linear units improve restricted boltzmann machines. In Int. Conf. Mach. Learn., pages 807-814, 2010.
[40′] Phan Ngoc Lan, Nguyen Sy An, Dao Viet Hang, Dao Van Long, Tran Quang Trung, Nguyen Thi Thuy, and Dinh Viet Sang. Neounet: Towards accurate colon polyp segmentation and neoplasm detection. In Adv. Vis. Comput.-Int. Symp., pages 15-28. Springer, 2021.
[41′] Ozan Oktay, Jo Schlemper, Loic Le Folgoc, Matthew Lee, Mattias Heinrich, Kazunari Misawa, Kensaku Mori, Steven McDonagh, Nils Y Hammerla, Bernhard Kainz, et al. Atten-tion u-net: Learning where to look for the pancreas. arXiv preprint arXiv: 1804.03999, 2018.
[42′] Md Mostafijur Rahman and Radu Marculescu. Medical image segmentation via cascaded attention decoding. In IEEE/CVF Winter Conf. Appl. Comput. Vis., pages 6222-6231, 2023.
[43′] Md Mostafijur Rahman and Radu Marculescu. Multi-scale hierarchical vision transformer with cascaded attention de-coding for medical image segmentation. In Med. Imaging Deep Learn., 2023.
[44′] Olaf Ronneberger, Philipp Fischer, and Thomas Brox. U-net: Convolutional networks for biomedical image segmentation. In Int. Conf. Med. Image Comput. Comput. Assist. Interv., pages 234-241. Springer, 2015.
[45′] Mark Sandler, Andrew Howard, Menglong Zhu, Andrey Zh-moginov, and Liang-Chieh Chen. Mobilenetv2: Inverted residuals and linear bottlenecks. In IEEE Conf. Comput. Vis. Pattern Recog., pages 4510-4520, 2018.
[46′] Karen Simonyan and Andrew Zisserman. Very deep convo-lutional networks for large-scale image recognition. arXiv preprint arXiv: 1409.1556, 2014.
[47′] Christian Szegedy, Wei Liu, Yangqing Jia, Pierre Sermanet, Scott Reed, Dragomir Anguelov, Dumitru Erhan, Vincent Vanhoucke, and Andrew Rabinovich. Going deeper with convolutions. In IEEE Conf. Comput. Vis. Pattern Recog., pages 1-9, 2015.
[48′] Mingxing Tan and Quoc Le. Efficientnet: Rethinking model scaling for convolutional neural networks. In Int. Conf. Mach. Learn., pages 6105-6114. PMLR, 2019.
[49′] Zhengzhong Tu, Hossein Talebi, Han Zhang, Feng Yang, Peyman Milanfar, Alan Bovik, and Yinxiao Li. Maxvit: Multi-axis vision transformer. In Eur. Conf. Comput. Vis., pages 459-479. Springer, 2022.
[50′] Jeya Maria Jose Valanarasu and Vishal M Patel. Unext: Mlp-based rapid medical image segmentation network. In Int. Conf. Med. Image Comput. Comput. Assist. Interv., pages 23-33. Springer, 2022.
[51′] David V'azquez, Jorge Bernal, F Javier S'anchez, Gloria Fernandez-Esparrach, Antonio M L'opez, Adriana Romero, Michal Drozdzal, and Aaron Courville. A benchmark for endoluminal scene segmentation of colonoscopy images. J. Healthc. Eng., 2017, 2017.
[52′] Haonan Wang, Peng Cao, Jiaqi Wang, and Osmar R Zaiane. Uctransnet: rethinking the skip connections in u-net from a channel-wise perspective with transformer. In AAAI, pages 2441-2449, 2022.
[53′] Hongyi Wang, Shiao Xie, Lanfen Lin, Yutaro Iwamoto, Xian-Hua Han, Yen-Wei Chen, and Ruofeng Tong. Mixed transformer u-net for medical image segmentation. In ICASSP, pages 2390-2394. IEEE, 2022.
[54′] Jinfeng Wang, Qiming Huang, Feilong Tang, Jia Meng, Jion-glong Su, and Sifan Song. Stepwise feature fusion: Local guides global. arXiv preprint arXiv: 2203.03635, 2022.
[55′] Wenhai Wang, Enze Xic, Xiang Li, Deng-Ping Fan, Kaitao Song, Ding Liang, Tong Lu, Ping Luo, and Ling Shao. Pyra-mid vision transformer: A versatile backbone for dense pre-diction without convolutions. In Int. Conf. Comput. Vis., pages 568-578, 2021.
[56′] Wenhai Wang, Enze Xie, Xiang Li, Deng-Ping Fan, Kaitao Song, Ding Liang, Tong Lu, Ping Luo, and Ling Shao. Pvt v2: Improved baselines with pyramid vision transformer. Comput. Vis. Media, 8 (3): 415-424, 2022.
[57′] Sanghyun Woo, Jongchan Park, Joon-Young Lee, and In So Kweon. Cham: Convolutional block attention module. In Eur. Conf. Comput. Vis., pages 3-19, 2018.
[58′] Enze Xie, Wenhai Wang, Zhiding Yu, Anima Anandkumar, Jose M Alvarez, and Ping Luo. Segformer: Simple and ef-ficient design for semantic segmentation with transformers. Adv. Neural Inform. Process. Syst., 34:12077-12090, 2021.
[59′] Weihao Yu, Mi Luo, Pan Zhou, Chenyang Si, Yichen Zhou, Xinchao Wang, Jiashi Feng, and Shuicheng Yan. Metaformer is actually what you need for vision. In IEEE Conf. Comput. Vis. Pattern Recog., pages 10819-10829, 2022.
[60′] Xiangyu Zhang, Xinyu Zhou, Mengxiao Lin, and Jian Sun. Shufflenet: An extremely efficient convolutional neural net-work for mobile devices. In IEEE Conf. Comput. Vis. Pattern Recog., pages 6848-6856, 2018.
[61′] Yundong Zhang, Huiye Liu, and Qiang Hu. Transfuse: Fus-ing transformers and cnns for medical image segmentation. In Int. Conf. Med. Image Comput. Comput. Assist. Interv., pages 14-24. Springer, 2021.
[62′] Zongwei Zhou, Md Mahfuzur Rahman Siddiquee, Nima Tajbakhsh, and Jianming Liang. Unet++: A nested u-net ar-chitecture for medical image segmentation. In Deep Learn. Med. Image Anal. Multimodal Learn. Clin. Decis. Support, pages 3-11. Springer, 2018.
[63′] Hu Cao, Yueyue Wang, Joy Chen, Dongsheng Jiang, Xiaopeng Zhang, Qi Tian, and ManningWang. Swin-unet: Unet-like pure transformer for medical image segmentation. arXiv preprint arXiv: 2105.05537, 2021.
[64′] Jieneng Chen, Yongyi Lu, Qihang Yu, Xiangde Luo, Ehsan Adeli, Yan Wang, Le Lu, Alan L Yuille, and Yuyin Zhou. Transunet: Transformers make strong encoders for medical image segmentation. arXiv preprint arXiv: 2102.04306, 2021.
[65′] Long Chen, Hanwang Zhang, Jun Xiao, Liqiang Nie, Jian Shao, Wei Liu, and Tat-Seng Chua. Sca-cnn: Spatial and channel-wise attention in convolutional networks for image captioning. In Proceedings of the IEEE Conference on Computer Vision and Pattern Recognition, pages 5659-5667, 2017.
[66′] Shuhan Chen, Xiuli Tan, Ben Wang, and Xuelong Hu. Reverse attention for salient object detection. In Proceedings of the European Conference on Computer Vision (ECCV), pages 234-250, 2018.
[67′] Xiangxiang Chu, Zhi Tian, Bo Zhang, Xinlong Wang, Xiaolin Wei, Huaxia Xia, and Chunhua Shen. Conditional positional encodings for vision transformers. arXiv preprint arXiv: 2102.10882, 2021.
[68′] Bo Dong, WenhaiWang, Deng-Ping Fan, Jinpeng Li, Huazhu Fu, and Ling Shao. Polyppvt: Polyp segmentation with pyramid vision transformers. arXiv preprint arXiv: 2108.06932, 2021.
[69′] Alexey Dosovitskiy, Lucas Beyer, Alexander Kolesnikov, Dirk Weissenborn, Xiaohua Zhai, Thomas Unterthiner, Mostafa Dehghani, Matthias Minderer, Georg Heigold, Sylvain Gelly, et al. An image is worth 16×16 words: Transformers for image recognition at scale. arXiv preprint arXiv: 2010.11929, 2020.
[70′] Deng-Ping Fan, Ge-Peng Ji, Tao Zhou, Geng Chen, Huazhu Fu, Jianbing Shen, and Ling Shao. Pranet: Parallel reverse attention network for polyp segmentation. In International Conference on Medical Image Computing and Computer-Assisted Intervention, pages 263-273. Springer, 2020.
[71′] Jie Hu, Li Shen, and Gang Sun. Squeeze-and-excitation networks. In Proceedings of the IEEE Conference on Computer Vision and Pattern Recognition, pages 7132-7141, 2018.
[72′] Huimin Huang, Lanfen Lin, Ruofeng Tong, Hongjie Hu, Qiaowei Zhang, Yutaro Iwamoto, Xianhua Han, Yen-Wei Chen, and Jian Wu. Unet 3+: A full-scale connected unet for medical image segmentation. In ICASSP 2020-2020 IEEE International Conference on Acoustics, Speech and Signal Processing (ICASSP), pages 1055-1059. IEEE, 2020.
[73′] Xiaohong Huang, Zhifang Deng, Dandan Li, and Xueguang Yuan. Missformer: An effective medical image segmentation transformer. arXiv preprint arXiv: 2109.07162, 2021.
[74′] Ailiang Lin, Bingzhi Chen, Jiayu Xu, Zheng Zhang, Guangming Lu, and David Zhang. Dstransunet: Dual swin transformer u-net for medical image segmentation. IEEE Transactions on Instrumentation and Measurement, 2022.
[75′] Ze Liu, Yutong Lin, Yue Cao, Han Hu, Yixuan Wei, Zheng Zhang, Stephen Lin, and Baining Guo. Swin transformer: Hierarchical vision transformer using shifted windows. In Proceedings of the IEEE/CVF International Conference on Computer Vision, pages 10012-10022, 2021.
[76′] Ilya Loshchilov and Frank Hutter. Decoupled weight decay regularization. arXiv preprint arXiv: 1711.05101, 2017.
[77′] Ange Lou, Shuyue Guan, and Murray Loew. Dc-unet: rethinking the u-net architecture with dual channel efficient enn for medical image segmentation. In Medical Imaging 2021: Image Processing, volume 11596, pages 758-768. SPIE, 2021.
[78′] Ozan Oktay, Jo Schlemper, Loic Le Folgoc, Matthew Lee, Mattias Heinrich, Kazunari Misawa, Kensaku Mori, Steven McDonagh, Nils Y Hammerla, Bernhard Kainz, et al. Attention u-net: Learning where to look for the pancreas. arXiv preprint arXiv: 1804.03999, 2018.
[79′] Md Mostafijur Rahman and Radu Marculescu. Medical image segmentation via cascaded attention decoding. In Proceedings of the IEEE/CVF Winter Conference on Applications of Computer Vision, pages 6222-6231, 2023.
[80′] Olaf Ronneberger, Philipp Fischer, and Thomas Brox. U-net: Convolutional networks for biomedical image segmentation. In International Conference on Medical Image Computing and Computer-Assisted Intervention, pages 234-241. Springer, 2015.
[81′] Hugo Touvron, Matthieu Cord, Matthijs Douze, Francisco Massa, Alexandre Sablayrolles, and Herv′e J′egou. Training data-efficient image transformers & distillation through attention. In International Conference on Machine Learning, pages 10347-10357. PMLR, 2021.
[82′] Zhengzhong Tu, Hossein Talebi, Han Zhang, Feng Yang, Peyman Milanfar, Alan Bovik, and Yinxiao Li. Maxvit: Multi-axis vision transformer. In Proceedings of the European Conference on Computer Vision (ECCV), pages 459-479. Springer, 2022.
[83′] Hongyi Wang, Shiao Xie, Lanfen Lin, Yutaro Iwamoto, Xian-Hua Han, Yen-Wei Chen, and Ruofeng Tong. Mixed transformer u-net for medical image segmentation. In ICASSP 2022-2022 IEEE International Conference on Acoustics, Speech and Signal Processing (ICASSP), pages 2390-2394. IEEE, 2022a.
[84′] Jinfeng Wang, Qiming Huang, Feilong Tang, Jia Meng, Jionglong Su, and Sifan Song. Stepwise feature fusion: Local guides global. arXiv preprint arXiv: 2203.03635, 2022b.
[85′] WenhaiWang, Enze Xie, Xiang Li, Deng-Ping Fan, Kaitao Song, Ding Liang, Tong Lu, Ping Luo, and Ling Shao. Pyramid vision transformer: A versatile backbone for dense prediction without convolutions. In Proceedings of the IEEE/CVF International Conference on Computer Vision, pages 568-578, 2021.
[86′] Wenhai Wang, Enze Xie, Xiang Li, Deng-Ping Fan, Kaitao Song, Ding Liang, Tong Lu, Ping Luo, and Ling Shao. Pvt v2: Improved baselines with pyramid vision transformer. Computational Visual Media, 8 (3): 415-424, 2022c.
[87′] Ross Wightman. Pytorch image models. https://github.com/rwightman/pytorch-image-models, 2019.
[88′] Sanghyun Woo, Jongchan Park, Joon-Young Lee, and In So Kweon. Cbam: Convolutional block attention module. In Proceedings of the European Conference on Computer Vision (ECCV), pages 3-19, 2018.
[89′] Enze Xie, Wenhai Wang, Zhiding Yu, Anima Anandkumar, Jose M Alvarez, and Ping Luo. Segformer: Simple and efficient design for semantic segmentation with transformers. Advances in Neural Information Processing Systems, 34:12077-12090, 2021.
[90′] Chenyu You, Ruihan Zhao, Fenglin Liu, Siyuan Dong, Sandeep P Chinchali, Lawrence Hamilton Staib, James s Duncan, et al. Class-aware adversarial transformers for medical image segmentation. In Advances in Neural Information Processing Systems, 2022.
[91′] Zongwei Zhou, Md Mahfuzur Rahman Siddiquee, Nima Tajbakhsh, and Jianming Liang. Unet++: A nested u-net architecture for medical image segmentation. In Deep learning in Medical Image Analysis and Multimodal Learning for Clinical Decision Support, pages 3-11. Springer, 2018.

What is claimed:

1. A method comprising:
receiving, by a processor, a set of one or more image data (e.g., image or video); and
determining, by the processor, a segmented region within at least one of the image data of the set of one or more image data using a deep neural network (e.g., CNN, CNN-based transformers) configured as a cascading transformer comprising (i) encoder configured with a plurality of encoding blocks arranged in a cascading manner and (ii) decoding blocks each comprising an attention gate that fuses features with skip connections from a corresponding encoder block and at least graph or multi-scale convolutional attention components,
wherein the segmented region or image data derived from the use of the segmented region is subsequently employed for diagnosis, controls, planning, assessment, or analysis.

2. The method of claim 1, wherein the decoding blocks, as a part of a graph convolutional decoder, are configured with the graph convolutional attention components (e.g., GCAM) employing at least one or more graph convolution layers.

3. The method of claim 2, wherein the graph convolutional attention component includes the at least one or more graph convolution layers connected to one or more convolution layers.

4. The method of claim 2, wherein each graph convolutional attention component includes a graph convolution block and a spatial attention module.

5. The method of claim 1, wherein, to aggregate the multi-scale features, each decoder block is configured to (i) upsample features from a previous decoder block with the features from a skip connection connected to the corresponding encoder block to generate combined upsampled features and (ii) direct the combined upsamples features to the decoding blocks, wherein each output of each stage of the decoding blocks are combined in a convolution layer (e.g., segmentation/prediction head).

6. The method of claim 1, wherein the decoding blocks, as a part of a multi-scale convolutional decoder, are configured with the multi-scale convolutional attention components (e.g., MSCAM) employing at least one or more multi-scale convolution layers.

7. The method of claim 6, wherein the multi-scale convolutional attention component includes the at least one or more multi-scale convolution layers connected to one or more convolution layers.

8. The method of claim 6, wherein each multi-scale convolutional attention component includes a multi-scale convolution block, a channel attention module, and a spatial attention module.

9. The method of claim 1, wherein, to aggregate the multi-scale features, each decoder block is configured to (i) upsample features from a previous decoder block with the features from a skip connection connected via a group attention gate (e.g., LGAG) to the corresponding encoder block to generate combined upsampled features and (ii) direct the combined upsamples features to the decoding blocks, wherein each output of each stage of the decoding blocks are combined in a convolution layer (e.g., segmentation/prediction head).

10. The method of claim 1, wherein the set of one or more image data comprises medical images (e.g., ultrasound, CT, MRI, endoscopy, OCT), and wherein the segmented region is subsequently employed for pretreatment diagnosis, treatment planning, and/or post-treatment assessments of a disease (e.g., to generate segmentation maps of lesions or organs).

11. The method of claim 1, wherein the deep neural network forms a hierarchical cascaded attention-based decoder.

12. The method of claim 1, wherein the segmented region or image data derived from use of the segmented region is employed in a control application (e.g., real-time control application) or for image analysis (e.g., in an image analysis toolkit).

13. The method of claim 1, wherein the set of one or more image data are 2D images, 3D objects (e.g., volumetric objects), or 4D images or objects (3D images or objects+time).

14. A system comprising:
a processor; and
a memory having instructions stored thereon, wherein execution of the instructions by the processor causes the processor to:
receive a set of one or more image data (e.g., image or video); and
determine a segmented region within at least one of the image data of the set of one or more image data using a deep neural network (e.g., CNN, CNN-based transformers) configured as a cascading transformer comprising (i) an encoder configured with a plurality of encoding blocks arranged in a cascading manner and (ii) decoding blocks each comprising an attention gate that fuses features with skip connections from a corresponding encoder block and at least graph or multi-scale convolutional attention components,
wherein the segmented region or image data derived from the use of the segmented region is subsequently employed for diagnosis, controls, planning, assessment, or analysis.

15. The system of claim 14, wherein the decoding blocks, as a part of a graph convolutional decoder, are configured with the graph convolutional attention components (e.g., GCAM) employing at least one or more graph convolution layers.

16. The system of claim 15, wherein the graph convolutional attention component includes the at least one or more graph convolution layers connected to one or more convolution layers.

17. The system of claim 15, wherein each graph convolutional attention component includes a graph convolution block and a spatial attention module.

18. The system of claim 14, wherein the decoding blocks, as a part of a multi-scale convolutional decoder, are configured with the multi-scale convolutional attention components (e.g., MSCAM) employing at least one or more multi-scale convolution layers.

19. The system of claim 18, wherein the multi-scale convolutional attention component includes the at least one or more multi-scale convolution layers connected to one or more convolution layers.

20. The system of claim 18, wherein each multi-scale convolutional attention component includes a multi-scale convolution block, a channel attention module, and a spatial attention module.

21. A non-transitory computer-readable medium having instructions stored thereon, wherein execution of the instructions by a processor causes the processor to:
receive a set of one or more image data (e.g., image or video); and
determine a segmented region within at least one of the image data of the set of one or more image data using a deep neural network (e.g., CNN, CNN-based, transformers) configured as a cascading transformer comprising (i) encoder configured with a plurality of encoding blocks arranged in a cascading manner and (ii) decoding blocks each comprising an attention gate that fuses features with skip connections from a corresponding encoder block and at least graph or multi-scale convolutional attention components,
wherein the segmented region or an image data derived from use of the segmented region is subsequently employed for diagnosis, controls, planning, assessment, or analysis.
